(19) United States
(12) Patent Application Publication
(10) Pub. No.: US 2021/0324369 A1
(43) Pub. Date: Oct. 21, 2021

(54) METHODS AND COMPOSITIONS FOR MESSENGER RNA PURIFICATION

(71) Applicant: Translate Bio, Inc., Lexington, MA (US)

(72) Inventors: Jonathan Abysalh, Lexington, MA (US); Jorel Vargas, Lexington, MA (US); Cameron M. Smith, Lexington, MA (US); Joseph Parrella, Lexington, MA (US); Frank DeRosa, Lexington, MA (US)

(21) Appl. No.: 17/172,527

(22) Filed: Feb. 10, 2021

Related U.S. Application Data
(60) Provisional application No. 62/972,471, filed on Feb. 10, 2020.

(51) Int. Cl. C12N 15/10

(52) U.S. Cl. CPC C12N 15/10

(57) ABSTRACT

The present invention provides, among other things, methods for purifying high quality messenger (mRNA) suitable for clinical use. The present invention is, in part, based on surprising discovery that capping and tailing mRNA in reaction buffer having a pH lower than 8.0 and MgCl₂ at a concentration of less than 1.25 mM can increase RNA integrity of final mRNA product. Thus, the present invention provides an effective, reliable, and efficient method of manufacturing high quality RNA at large scale for therapeutic use.

### CROSS-REFERENCE TO RELATED APPLICATIONS

This application claims priority to U.S. Provisional Application Ser. No. 62/972,471, filed Feb. 10, 2020, the disclosure of which is hereby incorporated by reference.

### BACKGROUND

Messenger RNA therapy (MRT) is a promising new approach to treat a variety of diseases. MRT involves administration of messenger RNA (mRNA) to a patient in need of the therapy. The administered mRNA produces a protein or peptide encoded by the mRNA within the patient's body. mRNA is typically synthesized using in vitro transcription systems (IVT) which involve enzymatic reactions by RNA polymerases. An IVT synthesis process is usually followed by reaction(s) for the addition of a 5′-cap (capping reaction) and a 3′-poly A tail (polyadenylation).

Effective mRNA therapy requires effective delivery of mRNA to the patient and efficient production of the protein encoded by the mRNA within the patient's body. To optimize mRNA delivery and protein production in vivo, a proper cap is typically required at the 5′ end of the construct, which protects the mRNA from degradation and facilitates successful protein translation. The presence of a “tail” at 3′ end serves to protect the mRNA from exonuclease degradation. New and improved methods are necessary to achieve mRNA at manufacturing scale for therapeutic use that results in high RNA integrity while maintaining high capping and tailing efficiency.

### SUMMARY OF THE INVENTION

The present invention provides an improved preparation method for in vitro transcribed (IVT) mRNA. The invention is based in part on the surprising discovery that capping and tailing mRNA in reaction conditions having a lower pH and lower concentration of magnesium chloride (MgCl₂) greatly improves RNA integrity of the mRNA product while maintaining all other critical quality attributes. Specifically, a capping and tailing reaction condition disclosed herein can successfully reduce degraded RNA species in the final mRNA product. This unique and advantageous condition of capping and tailing reaction condition was not appreciated prior to the present invention and is truly unexpected especially because the optimized cap and tail condition is able to increase the RNA integrity of mRNA product by at least about 25%. Based on this unexpected discovery, the present inventors have successfully developed a large-scale production method to synthesize and purify mRNA molecules that have high RNA integrity suitable for mRNA therapeutics. Thus, the present invention permits more efficient and reliable manufacturing of mRNA for therapeutic use.

In one aspect, the invention provides a method of capping and tailing an in vitro transcribed purified messenger RNA (mRNA) preparation, the method comprising capping and tailing the mRNA in a reaction buffer comprising MgCl₂ and having a pH lower than 8.0.

In some embodiments, the method comprises capping the mRNA in a reaction buffer comprising MgCl₂ and having a pH lower than 8.0. In some embodiments, the method comprises tailing the mRNA in a reaction buffer comprising MgCl₂ and having a pH lower than 8.0. Typically, the step of capping the mRNA in a reaction buffer comprising MgCl₂ and having a pH lower than 8 and the step of tailing the mRNA in a reaction buffer comprising MgCl₂ and having a pH lower than 8.0 are performed separately. In some embodiments, the step of capping the mRNA in a reaction buffer comprising MgCl₂ and having a pH lower than 8 and the step of tailing the mRNA in a reaction buffer comprising MgCl₂ and having a pH lower than 8.0 are performed sequentially.

In some embodiments, the reaction buffer further comprises salt. In some embodiments, the reaction buffer further comprises KCl. In some embodiments, the reaction buffer further comprises NaCl. In some embodiments, the reaction buffer further comprises CaCl₂. In some embodiments, the reaction buffer further comprises LiCl. In some embodiments, the reaction buffer further comprises ammonium acetate. In some embodiments, the reaction buffer further comprises a combination of salts. In some embodiment, the reaction buffer comprises salt at a concentration ranging from 0.1 mM to 100 mM. In some embodiment, the reaction buffer comprises salt at a concentration ranging from 1 mM to 50 mM. In some embodiment, the reaction buffer comprises salt at a concentration ranging from 1 mM to 10 mM. In some embodiment, the reaction buffer comprises salt at a concentration ranging from 5 mM to 8 mM. In some embodiment, the reaction buffer comprises salt at a concentration of 1 mM. In some embodiment, the reaction buffer comprises salt at a concentration of 3 mM. In some embodiment, the reaction buffer comprises salt at a concentration of 5 mM. In some embodiment, the reaction buffer comprises salt at a concentration of 8 mM. In some embodiment, the reaction buffer comprises salt at a concentration of 10 mM.

In some embodiments, the MgCl₂ in the reaction buffer has a concentration of about between 0.10 mM and 1.25. In some embodiments, the MgCl₂ in the reaction buffer has a concentration of about between 0.75 mM and 1.25 mM. In some embodiments, the MgCl₂ in the reaction buffer has a concentration of about between 0.50 mM and 1.0 mM. In some embodiments, the MgCl₂ in the reaction buffer has a concentration of about between 0.75 mM and 1.0 mM. In some embodiments, the MgCl₂ in the reaction buffer has a concentration of 0.25 mM. In some embodiments, the MgCl₂ in the reaction buffer has a concentration of 0.5 mM. In some embodiments, the MgCl₂ in the reaction buffer has a concentration of 0.7 mM. In some embodiments, the MgCl₂ in the reaction buffer has a concentration of 0.75 mM. In some embodiments, the MgCl₂ in the reaction buffer has a concentration of 0.8 mM. In some embodiments, the MgCl₂ in the reaction buffer has a concentration of 0.9 mM. In some embodiments, the MgCl₂ in the reaction buffer has a concentration of 1.0 mM. In some embodiments, the MgCl₂ in the reaction buffer has a concentration of 1.10 mM. In some embodiments, the MgCl₂ in the reaction buffer has a concentration of 1.20 mM.

In some embodiments, the reaction buffer comprises MnCl₂. In some embodiments, the reaction buffer comprises MgCl₂ and MnCl₂.

In some embodiments, the MnCl₂ in the reaction buffer has a concentration of about between 0.10 mM and 1.25. In some embodiments, the MnCl₂ in the reaction buffer has a concentration of about between 0.75 mM and 1.25 mM. In some embodiments, the MnCl₂ in the reaction buffer has a concentration of about between 0.50 mM and 1.0 mM. In some embodiments, the MnCl₂ in the reaction buffer has a concentration of about between 0.75 mM and 1.0 mM. In some embodiments, the MnCl₂ in the reaction buffer has a concentration of 0.25 mM. In some embodiments, the MnCl₂ in the reaction buffer has a concentration of 0.5 mM. In some embodiments, the MnCl₂ in the reaction buffer has a concentration of 0.7 mM. In some embodiments, the MnCl₂ in the reaction buffer has a concentration of 0.75 mM. In some embodiments, the MnCl₂ in the reaction buffer has a concentration of 0.8 mM. In some embodiments, the MnCl₂ in the reaction buffer has a concentration of 0.9 mM. In some embodiments, the MnCl₂ in the reaction buffer has a concentration of 1.0 mM. In some embodiments, the MnCl₂ in the reaction buffer has a concentration of 1.10 mM. In some embodiments, the MnCl₂ in the reaction buffer has a concentration of 1.20 mM.

In some embodiments, the pH of the reaction buffer is between about 6.0 and 8.0. In some embodiments, the pH of the reaction buffer is between about 6.5 and 8.0. In some embodiments, the pH of the reaction buffer is between about 7.0 and 7.8. In some embodiments, the pH of the reaction buffer is between about 7.2 and 7.7. In some embodiments, the pH of the reaction buffer is between about 7.4 and 7.6. In some embodiments, the pH of the reaction buffer is about 7.0. In some embodiments, the pH of the reaction buffer is about 7.2. In some embodiments, the pH of the reaction buffer is about 7.3. In some embodiments, the pH of the reaction buffer is about 7.4. In some embodiments, the pH of the reaction buffer is about 7.5. In some embodiments, the pH of the reaction buffer is about 7.6. In some embodiments, the pH of the reaction buffer is about 7.7. In some embodiments, the pH of the reaction buffer is about 7.8. In some embodiments, the pH of the reaction buffer is about 8.0.

In some embodiments, the mRNA is at a scale of 5 mg, 1 g, 15 g, 100 g, 250 g, 500 g, or 1 kg or above. In some embodiments, a method according to the invention results in mRNA of at least 100 mg, 150 mg, 200 mg, 300 mg, 400 mg, 500 mg, 600 mg, 700 mg, 800 mg, 900 mg, 1 g, 5 g, 10 g, 25 g, 50 g, 75 g, 100 g, 250 g, 500 g, 750 g, 1 kg, 5 kg, 10 kg, 50 kg, 100 kg, 1000 kg, or more at a single batch. In some embodiments, a method according to the invention results in mRNA of at least 5 mg at a single batch. In some embodiments, a method according to the invention results in mRNA of at least 100 mg at a single batch. In some embodiments, a method according to the invention results in mRNA of at least 500 mg at a single batch. In some embodiments, a method according to the invention results in mRNA of at least 1 g at a single batch. In some embodiments, a method according to the invention results in mRNA of at least 5 g at a single batch. In some embodiments, a method according to the invention results in mRNA of at least 10 g at a single batch. In some embodiments, a method according to the invention results in mRNA of at least 15 g at a single batch. In some embodiments, a method according to the invention results in mRNA of at least 50 g at a single batch. In some embodiments, a method according to the invention results in mRNA of at least 100 g at a single batch. In some embodiments, a method according to the invention results in mRNA of at least 250 g at a single batch. In some embodiments, a method according to the invention results in mRNA of at least 500 g at a single batch. In some embodiments, a method according to the invention results in mRNA of at least 1 kg at a single batch. In some embodiments, a method according to the invention results in mRNA of at least 10 kg at a single batch. In some embodiments, a method according to the invention results in mRNA of at least 50 kg at a single batch. In some embodiments, a method according to the invention results in mRNA of at least 100 kg at a single batch. As used herein, the term “batch” refers to a quantity or amount of mRNA synthesized at one time, e.g., produced according to a single manufacturing setting. A batch may refer to an amount of mRNA synthesized in one reaction that occurs via a single aliquot of enzyme and/or a single aliquot of DNA template for continuous synthesis under one set of conditions. mRNA synthesized at a single batch would not include mRNA synthesized at different times that are combined to achieve the desired amount.

In some embodiments, the tailing the mRNA comprises addition of a poly-A tail having a length of about between 50 nucleotides and 1000 nucleotides. In some embodiments, the tailing the mRNA comprises addition of a poly-A tail having a length of about between 100 nucleotides and 900 nucleotides. In some embodiments, the tailing the mRNA comprises addition of a poly-A tail having a length of about between 250 nucleotides and 750 nucleotides. In some embodiments, the tailing the mRNA comprises addition of a poly-A tail having a length of greater than about 50 nucleotides. In some embodiments, the tailing the mRNA comprises addition of a poly-A tail having a length of greater than about 100 nucleotides. In some embodiments, the tailing the mRNA comprises addition of a poly-A tail having a length of greater than about 200 nucleotides. In some embodiments, the tailing the mRNA comprises addition of a poly-A tail having a length of greater than about 250 nucleotides. In some embodiments, the tailing the mRNA comprises addition of a poly-A tail having a length of greater than about 300 nucleotides. In some embodiments, the tailing the mRNA comprises addition of a poly-A tail having a length of greater than about 400 nucleotides. In some embodiments, the tailing the mRNA comprises addition of a poly-A tail having a length of greater than about 500 nucleotides. In some embodiments, the tailing the mRNA comprises addition of a poly-A tail having a length of greater than about 600 nucleotides. In some embodiments, the tailing the mRNA comprises addition of a poly-A tail having a length of greater than about 750 nucleotides. In some embodiments, the tailing the mRNA comprises addition of a poly-A tail having a length of greater than about 900 nucleotides. In some embodiments, the tailing the mRNA comprises addition of a poly-A tail having a length of about 250 nucleotides. In some embodiments, the tailing the mRNA comprises addition of a poly-A tail having a length of about 500 nucleotides. In some embodiments, the tailing the mRNA comprises addition of a poly-A tail having a length of about 600 nucleotides. In some embodiments, the tailing the mRNA comprises addition of a poly-A tail having a length of about 700 nucleotides. In some embodiments, the tailing the mRNA comprises addition of a poly-A tail having a length of about 750 nucleotides. In some embodiments, the tailing the mRNA comprises addition of a poly-A tail having a length of about 900 nucleotides.

In some embodiments, tailing the mRNA has an efficiency of between about 70% and 95%. In some embodiments, tailing the mRNA has an efficiency of greater than about 60%. In some embodiments, tailing the mRNA has an efficiency of greater than about 70%. In some embodiments, tailing the mRNA has an efficiency of greater than about 72%. In some embodiments, tailing the mRNA has an efficiency of greater than about 75%. In some embodiments, tailing the mRNA has an efficiency of greater than about 78%. In some embodiments, tailing the mRNA has an efficiency of greater than about 80%. In some embodiments, tailing the mRNA has an efficiency of greater than about 82%. In some embodiments, tailing the mRNA has an efficiency of greater than about 85%. In some embodiments, tailing the mRNA has an efficiency of greater than about 88%. In some embodiments, tailing the mRNA has an efficiency of greater than about 90%. In some embodiments, tailing the mRNA has an efficiency of greater than about 95%. In some embodiments, tailing the mRNA has an efficiency of greater than about 97%. In some embodiments, tailing the mRNA has an efficiency of greater than about 99%. In some embodiments, tailing the mRNA has an efficiency of about 70%. In some embodiments, tailing the mRNA has an efficiency of about 72%. In some embodiments, tailing the mRNA has an efficiency of about 75%. In some embodiments, tailing the mRNA has an efficiency of about 78%. In some embodiments, tailing the mRNA has an efficiency of about 80%. In some embodiments, tailing the mRNA has an efficiency of about 82%. In some embodiments, tailing the mRNA has an efficiency of about 85%. In some embodiments, tailing the mRNA has an efficiency of about 88%. In some embodiments, tailing the mRNA has an efficiency of about 90%. In some embodiments, tailing the mRNA has an efficiency of about 95%. In some embodiments, tailing the mRNA has an efficiency of about 97%. In some embodiments, tailing the mRNA has an efficiency of about 99%. In some embodiments, tailing the mRNA has an efficiency of about 100%. In some embodiments, the tailing efficiency is assessed by Capillary Electrophoresis (CE) shift.

In some embodiments, capping and tailing the mRNA in a reaction buffer having a pH lower than 8.0 results in capped and tailed mRNA that has greater integrity in comparison to capped and tailed mRNA using a reaction buffer having a pH of 8.0 or above.

In some embodiments, capping and tailing the mRNA in a reaction buffer having a MgCl₂ concentration of 1.0 mM or less results in a capped and tailed mRNA that has greater integrity in comparison to capped and tailed mRNA using a reaction buffer having a MgCl₂ concentration of greater than 1.0 mM.

In some embodiments, the mRNA integrity is at least 60% or more. In some embodiments, the mRNA integrity is at least 65% or more. In some embodiments, the mRNA integrity is at least 70% or more. In some embodiments, the mRNA integrity is at least 75% or more. In some embodiments, the mRNA integrity is at least 80% or more. In some embodiments, the mRNA integrity is at least 85% or more. In some embodiments, the mRNA integrity is at least 90% or more. In some embodiments, the mRNA integrity is at least 92% or more. In some embodiments, the mRNA integrity is at least 95% or more. In some embodiments, the mRNA integrity is at least 99% or more. In some embodiments, the mRNA integrity is assessed by Capillary Electrophoresis (CE) smear. In some embodiments, the mRNA integrity is assessed by CGE smear.

In some embodiments, the method has an mRNA capping efficiency of 70% or above. In some embodiments, the method has an mRNA capping efficiency of 80% or above. In some embodiments, the method has an mRNA capping efficiency of 85% or above. In some embodiments, the method has an mRNA capping efficiency of 90% or above. In some embodiments, the method has an mRNA capping efficiency of 95% or above. In some embodiments, the method has an mRNA capping efficiency of 98% or above. In some embodiments, the method has an mRNA capping efficiency of 80%. In some embodiments, the method has an mRNA capping efficiency of 85%. In some embodiments, the method has an mRNA capping efficiency of 90%. In some embodiments, the method has an mRNA capping efficiency of 95%. In some embodiments, the method has an mRNA capping efficiency of 97%. In some embodiments, the method has an mRNA capping efficiency of 98%. In some embodiments, the method has an mRNA capping efficiency of 99%. In some embodiments, the method has an mRNA capping efficiency of 100%.

In one aspect, the present invention provides, among other things, a method of capping and tailing an in vitro transcribed purified messenger RNA (mRNA) preparation, the method comprising capping and tailing the mRNA in a reaction buffer comprising a pH of about 7.5, and a MgCl₂ concentration of about 1.0 mM, wherein the capping and tailing of the mRNA has a capping and tailing efficiency of 80% or more, and wherein the capped and tailed mRNA has an integrity of at least 65% or above.

### BRIEF DESCRIPTION OF THE DRAWINGS

The above and further features will be more clearly appreciated from the following detailed description when taken in conjunction with the accompanying drawings. The drawings however are for illustration purposes only; not for limitation.

FIG. 1 shows capillary electrophoresis (CE) profiles of purified CFTR mRNA, prior to capping and tailing (left graph), purified capped and tailed CFTR mRNA in reaction buffer comprising 1.25 mM MgCl₂ at pH 8.0 (middle graph) and purified capped and tailed CFTR mRNA in reaction buffer comprising 1.0 mM MgCl₂ at pH 7.5 (right graph) at 5 mg scale. The arrow indicates a shoulder, which represents degraded RNA species.

FIG. 2 shows capillary electrophoresis (CE) profiles of purified DNAH5 mRNA, prior to capping and tailing (left graph), purified capped and tailed CFTR mRNA in reaction buffer comprising 1.25 mM MgCl₂ at pH 8.0 (middle graph) and purified capped and tailed CFTR mRNA in reaction buffer comprising 1.0 mM MgCl₂ at pH 7.5 (right graph) at 5 mg scale. The arrow indicates a shoulder, which represents degraded RNA species.

FIG. 3 shows capillary electrophoresis (CE) profile of purified capped and tailed CFTR mRNA in reaction buffer comprising 1.0 mM MgCl₂ at pH 7.5 at 1-gram scale, demonstrating the integrity of the mRNA capped and tailed in an optimized reaction condition. The arrow indicates a shoulder, which represents degraded RNA species.

FIG. 4 shows capillary electrophoresis (CE) profiles of purified CFTR mRNA, prior to capping and tailing (left graph), purified capped and tailed CFTR mRNA in reaction buffer comprising 1.25 mM MgCl₂ at pH 8.0 (middle graph) and purified capped and tailed CFTR mRNA in reaction buffer comprising 1.0 mM MgCl₂ at pH 7.5 (right graph) at 15-gram scale. The arrow indicates a shoulder, which represents degraded RNA species.

FIG. 5 shows capillary electrophoresis (CE) profiles of purified CFTR mRNA, prior to capping and tailing (left graph), purified capped and tailed CFTR mRNA in reaction buffer comprising 1.25 mM MgCl₂ at pH 8.0 (middle graph) and purified capped and tailed CFTR mRNA in reaction buffer comprising 1.0 mM MgCl₂ at pH 7.5 (right graph) at 100-gram manufacturing scale. The arrow indicates a shoulder, which represents degraded RNA species.

FIG. 6 shows capillary electrophoresis (CE) profiles of purified capped and tailed OTC mRNA in reaction buffer comprising 1.25 mM MgCl₂ at pH 8.0 (left graph) at 10-gram manufacturing scale and purified capped and tailed OTC mRNA in reaction buffer comprising 1.0 mM MgCl₂ at pH 7.5 (right graph) at 250-gram manufacturing scale. The arrow indicates a shoulder, which represents degraded RNA species.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Amino acid: As used herein, the term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Batch: As used herein, the term “batch” refers to a quantity or amount of mRNA synthesized at one time, e.g., produced according to a single manufacturing order during the same cycle of manufacture. A batch may refer to an amount of mRNA synthesized in one reaction that occurs via a single aliquot of enzyme and/or a single aliquot of DNA template for continuous synthesis under one set of conditions. In some embodiments, a batch would include the mRNA produced from a reaction in which not all reagents and/or components are supplemented and/or replenished as the reaction progresses. The term “batch” would not mean mRNA synthesized at different times that are combined to achieve the desired amount.

Biologically active: As used herein, the term “biologically active” refers to a characteristic of any agent that has activity in a biological system, and particularly in an organism. For instance, an agent that, when administered to an organism, has a biological effect on that organism, is considered to be biologically active.

Codon optimization: As used herein, the terms “codon optimization” and “codon-optimized” refer to modifications of the codon composition of a naturally-occurring or wild-type nucleic acid encoding a peptide, polypeptide or protein that do not alter its amino acid sequence, thereby improving protein expression of said nucleic acid. Such modifications to the naturally-occurring or wild-type nucleic acid may be done to achieve the highest possible G/C content, to adjust codon usage to avoid rare or rate-limiting codons, to remove destabilizing nucleic acid sequences or motifs and/or to eliminate pause sites or terminator sequences.

Contaminants: As used herein, the term “contaminants” refers to substances inside a confined amount of liquid, gas, or solid, which differ from the chemical composition of the target material or compound. Contaminants are also referred to as impurities. Examples of contaminants or impurities include buffers, proteins (e.g., enzymes), nucleic acids, salts, solvents, and/or wash solutions.

Dispersant: As used herein, the term “dispersant” refers to a solid particulate which reduces the likelihood that an mRNA precipitate will form a hydrogel. Examples of dispersants include and are not limited to one or more of ash, clay, diatomaceous earth, filtering agent, glass beads, plastic beads, polymers, polypropylene beads, polystyrene beads, salts (e.g., cellulose salts), sand, and sugars. In embodiments, a dispersant is polymer microspheres (e.g., poly(styrene-co-divinylbenezene) microspheres).

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery). In some embodiments, delivery is pulmonary delivery, e.g., comprising nebulization.

Encapsulation: As used herein, the term “encapsulation,” or its grammatical equivalent, refers to the process of confining a nucleic acid molecule within a nanoparticle.

Expression: As used herein, “expression” of a nucleic acid sequence refers to translation of an mRNA into a polypeptide, assemble multiple polypeptides (e.g., heavy chain or light chain of antibody) into an intact protein (e.g., antibody) and/or post-translational modification of a polypeptide or fully assembled protein (e.g., antibody). In this application, the terms “expression” and “production,” and their grammatical equivalents, are used interchangeably.

Full-length mRNA: As used herein, “full-length mRNA” is as characterized when using a specific assay, e.g., gel electrophoresis or detection using UV and UV absorption spectroscopy with separation by capillary electrophoresis. The length of an mRNA molecule that encodes a full-length polypeptide and as obtained following any of the purification methods described herein is at least 50% of the length of a full-length mRNA molecule that is transcribed from the target DNA, e.g., at least 60%, 70%, 80%, 90%, 91%, 92%, 93%, 94%, 95%, 96%, 97%, 98%, 99%, 99.01%, 99.05%, 99.1%, 99.2%, 99.3%, 99.4%, 99.5%, 99.6%, 99.7%, 99.8%, 99.9% of the length of a full-length mRNA molecule that is transcribed from the target DNA and prior to purification according to any method described herein.

Functional: As used herein, a “functional” biological molecule is a biological molecule in a form in which it exhibits a property and/or activity by which it is characterized.

Half-life: As used herein, the term “half-life” is the time required for a quantity such as nucleic acid or protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

Liposome: As used herein, the term “liposome” refers to any lamellar, multilamellar, or solid nanoparticle vesicle. Typically, a liposome as used herein can be formed by mixing one or more lipids or by mixing one or more lipids and polymer(s). In some embodiments, a liposome suitable for the present invention contains a cationic lipids(s) and optionally non-cationic lipid(s), optionally cholesterol-based lipid(s), and/or optionally PEG-modified lipid(s).

messenger RNA (mRNA): As used herein, the term “messenger RNA (mRNA)” refers to a polynucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated.

mRNA Integrity: As used herein, the term “mRNA integrity” generally refers to the quality of mRNA. In some embodiments, mRNA integrity refers to the percentage of mRNA that is not degraded after a purification process (e.g., a method described herein). mRNA integrity may be determined using methods particularly described herein, such as TAE Agarose gel electrophoresis or by SDS-PAGE with silver staining, or by methods well known in the art, for example, by RNA agarose gel electrophoresis (e.g., Ausubel et al., John Wiley & Sons, Inc., 1997, Current Protocols in Molecular Biology).

N/P Ratio: As used herein, the term “N/P ratio” refers to a molar ratio of positively charged molecular units in the cationic lipids in a lipid nanoparticle relative to negatively charged molecular units in the mRNA encapsulated within that lipid nanoparticle. As such, N/P ratio is typically calculated as the ratio of moles of amine groups in cationic lipids in a lipid nanoparticle relative to moles of phosphate groups in mRNA encapsulated within that lipid nanoparticle.

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguano sine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified in order to facilitate or achieve delivery. In some embodiments, the nucleotides T and U are used interchangeably in sequence descriptions.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In specific embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al. describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, sulfonate and aryl sulfonate. Further pharmaceutically acceptable salts include salts formed from the quarternization of an amine using an appropriate electrophile, e.g., an alkyl halide, to form a quarternized alkylated amino salt.

Systemic distribution or delivery: As used herein, the terms “systemic distribution,” “systemic delivery,” or grammatical equivalent, refer to a delivery or distribution mechanism or approach that affect the entire body or an entire organism. Typically, systemic distribution or delivery is accomplished via body's circulation system, e.g., blood stream. Compared to the definition of “local distribution or delivery.”

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

### DETAILED DESCRIPTION

The present invention relates to methods for preparing scalable quantities of pure and high-quality mRNA. mRNA is typically synthesized by in vitro transcription (IVT) using polymerases such as SP6 or T7-polymerase, then capped and tailed to generate the full length in vivo translatable mRNA. A preparation of correctly capped RNAs is essential to assess the function of mRNAs in the cellular context. Furthermore, altering the cap structure bears potential to increase mRNA stability and translational efficiency—two properties which may provide the key to therapeutic applications of mRNA.

The present inventions is based, at least in part, on a surprising and unexpected discovery that when a buffer having a pH lower than the conventional pH and comprising lower concentration of magnesium chloride (MgCl₂) was used in capping and tailing reaction, RNA integrity improved by more than 25% as assessed by capillary electrophoresis (CE) or capillary gel electrophoresis (CGE). The mRNA preparation method disclosed in herein also maintained high capping and tailing efficiencies. This improvement was translatable across different scales, demonstrating scalability of the method and suitability for use in mRNA manufacturing and therapeutics.

### 5-Cap

Typically, eukaryotic mRNAs bear a “cap” structure at their 5′-termini, which plays an important role in translation. For example, the cap plays a pivotal role in mRNA metabolism, and is required to varying degrees for processing and maturation of an RNA transcript in the nucleus, transport of mRNA from the nucleus to the cytoplasm, mRNA stability, and efficient translation of the mRNA to protein. The 5′ cap structure is involved in the initiation of protein synthesis of eukaryotic cellular and eukaryotic viral mRNAs and in mRNA processing and stability in vivo (see, e.g, Shatkin, A. J., CELL, 9: 645-653 (1976); Furuichi, et al., NATURE, 266: 235 (1977); FEDERATION OF EXPERIMENTAL BIOLOGISTS SOCIETY LETTER 96: 1-11 (1978); Sonenberg, N., PROG. NUC. ACID RES MOL BIOL, 35: 173-207 (1988)). Specific cap binding proteins exist that are components of the machinery required for initiation of translation of an mRNA (see, e.g., Shatkin, A. J., CELL, 40: 223-24 (1985); Sonenberg, N., PROG. NUC. ACID RES MOL BIOL, 35: 173-207 (1988)). The cap of mRNA is recognized by the translational initiation factor eIF4E (Gingras, et al., ANN. REV. BIOCHEM. 68: 913-963 (1999); Rhoads, R. E., J. BIOL. CHEM. 274: 30337-3040 (1999)). The 5′ cap structure also provides resistance to 5′-exonuclease activity and its absence results in rapid degradation of the mRNA (see, e.g., Ross, J., MOL. BIOL. MED. 5: 1-14 (1988); Green, M. R. et al., CELL, 32: 681-694 (1983)). Since the primary transcripts of many eukaryotic cellular genes and eukaryotic viral genes require processing to remove intervening sequences (introns) within the coding regions of these transcripts, the benefit of the cap also extends to stabilization of such pre-mRNA.

In vitro, capped RNAs have been reported to be translated more efficiently than uncapped transcripts in a variety of in vitro translation systems, such as rabbit reticulocyte lysate or wheat germ translation systems (see, e.g., Shimotohno, K., et al., PROC. NATL. ACAD. SCI. USA, 74: 2734-2738 (1977); Paterson and Rosenberg, NATURE, 279: 692 (1979)). This effect is also believed to be due in part to protection of the RNA from exoribonucleases present in the in vitro translation system, as well as other factors.

Naturally occurring cap structures comprise a 7-methyl guanosine that is linked via a triphosphate bridge to the 5′-end of the first transcribed nucleotide, resulting in a dinucleotide cap of m⁷G(5′)ppp(5′)N, where N is any nucleoside. In vivo, the cap is added enzymatically. The cap is added in the nucleus and is catalyzed by the enzyme guanylyl transferase. The addition of the cap to the 5′ terminal end of RNA occurs immediately after initiation of transcription. The terminal nucleoside is typically a guanosine, and is in the reverse orientation to all the other nucleotides, i.e., G(5′)ppp(5′)GpNpNp.

A common cap for mRNA produced by in vitro transcription is m7G(5′)ppp(5′)G, which has been used as the dinucleotide cap in transcription with T7 or SP6 RNA polymerase in vitro to obtain RNAs having a cap structure in their 5′-termini. The prevailing method for the in vitro synthesis of capped mRNA employs a pre-formed dinucleotide of the form m7G(5′)ppp(5′)G (“m7GpppG”) as an initiator of transcription. A disadvantage of using m7G(5′)ppp(5′)G, a pseudosymmetrical dinucleotide, is the propensity of the 3′-OH of either the G or m7G moiety to serve as the initiating nucleophile for transcriptional elongation. In other words, the presence of a 3′-OH on both the m7G and G moieties leads to up to half of the mRNAs incorporating caps in an improper orientation. This leads to the synthesis of two isomeric RNAs of the form m7G(5′)pppG(pN)n and G(5′)pppm7G(pN)n, in approximately equal proportions, depending upon the ionic conditions of the transcription reaction. Variations in the isomeric forms can adversely effect in vitro translation and are undesirable for a homogenous therapeutic product.

To date, the usual form of a synthetic dinucleotide cap used in in vitro translation experiments is the Anti-Reverse Cap Analog (“ARCA”), which is generally a modified cap analog in which the 2′ or 3′ OH group is replaced with —OCH₃. ARCA and triple-methylated cap analogs are incorporated in the forward orientation. Chemical modification of m⁷G at either the 2′ or 3′ OH group of the ribose ring results in the cap being incorporated solely in the forward orientation, even though the 2′ OH group does not participate in the phosphodiester bond. (Jemielity, J. et al., “Novel ‘anti-reverse’ cap analogs with superior translational properties”, RNA, 9: 1108-1122 (2003)). The selective procedure for methylation of guanosine at N7 and 3′ O-methylation and 5′ diphosphate synthesis has been established (Kore, A. and Parmar, G. NUCLEOSIDES, NUCLEOTIDES, AND NUCLEIC ACIDS, 25:337-340, (2006) and Kore, A. R., et al. NUCLEOSIDES, NUCLEOTIDES, AND NUCLEIC ACIDS 25(3): 307-14, (2006).

Transcription of RNA usually starts with a nucleoside triphosphate (usually a purine, A or G). In vitro transcription typically comprises a phage RNA polymerase such as T7, T3 or SP6, a DNA template containing a phage polymerase promoter, nucleotides (ATP, GTP, CTP and UTP) and a buffer containing magnesium salt. The synthesis of capped RNA includes the incorporation of a cap analog (e.g., m7GpppG) in the transcription reaction, which in some embodiments is incorporated by the addition of recombinant guanylyl transferase. Excess m7GpppG to GTP (4:1) increases the opportunity that each transcript will have a 5′ cap. Kits for capping of in vitro transcribed mRNAs are commercially available, including the mMESSAGE mMACHINE® kit (Ambion, Inc., Austin, Tex.). These kits will typically yield 80% capped RNA to 20% uncapped RNA, although total RNA yields are lower as GTP concentration becomes rate limiting as GTP is needed for the elongation of the transcript. On the other hand, the methods described herein yields capping efficiency greater than 90% and RNA integrity of greater than 70%.

In some embodiments, inventive methods of the present invention can be used to add a cap having a structure of formula I:

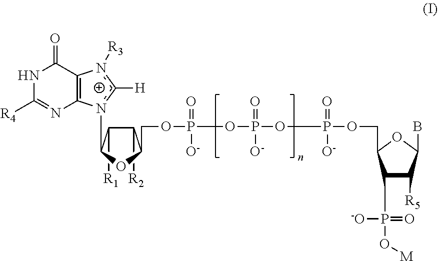

wherein,
B is a nucleobase;
R₁ is selected from a halogen, OH, and OCH₃;
R₂ is selected from H, OH, and OCH₃;
R₃ is CH₃, CH₂CH₃, CH₂CH₂CH₃ or void;
R₄ is NH₂;
R₅ is selected from OH, OCH₃ and a halogen;
n is 1, 2, or 3; and
M is a nucleotide of the mRNA.

In some embodiments, the nucleobase is guanine.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. Additional cap structures are described in published U.S. Application No. US 2016/0032356 and published U.S. Application No. US 2018/0125989, which are incorporated herein by reference.

### 3′-Poly A Tail

The presence of a “tail” at 3′ end serves to protect the mRNA from exonuclease degradation. The 3′ tail may be added before, after or at the same time of adding the 5′ Cap.

In some embodiments, the poly A tail is 25-5,000 nucleotides in length. Typically, a tail structure includes a poly A and/or poly C tail. (A, adenosine; C, cytosine). In some embodiments, a poly-A or poly-C tail on the 3′ terminus of mRNA includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively. In some embodiments, a poly-A or poly-C tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively. In some embodiments, a tail structure includes is a combination of poly A and poly C tails with various lengths described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

Other capping and/or tailing methods are available in the art and may be used to practice the present invention.

As described herein, the addition of the 5′ cap and/or the 3′ tail facilitates the detection of abortive transcripts generated during in vitro synthesis because without capping and/or tailing, the size of those prematurely aborted mRNA transcripts can be too small to be detected. Thus, in some embodiments, the 5′ cap and/or the 3′ tail are added to the synthesized mRNA before the mRNA is tested for purity (e.g., the level of abortive transcripts present in the mRNA). In some embodiments, the 5′ cap and/or the 3′ tail are added to the synthesized mRNA before the mRNA is purified as described herein. In other embodiments, the 5′ cap and/or the 3′ tail are added to the synthesized mRNA after the mRNA is purified as described herein.

mRNA Synthesis and Purification

The maintenance of high RNA integrity during the in vitro transcription synthesis and mRNA purification is critical in manufacturing mRNA for therapeutic purpose. Additionally, high capping and tailing efficiency of mRNA with poly A tail of desired length are important attributes of mRNA quality. mRNAs according to the present invention may be synthesized according to any of a variety of known methods. Various methods are described in published U.S. Application No. US 2018/0258423, and can be used to practice the present invention, all of which are incorporated herein by reference. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions will vary according to the specific application.

In some embodiments, the in vitro transcription occurs in a single batch. In some embodiments, IVT reaction includes capping and tailing reactions (C/T). In some embodiments, capping and tailing reactions are performed separately from IVT reaction. In some embodiments, the mRNA is recovered from IVT reaction, followed by a first precipitation and purification of mRNA by methods known in the art; the recovered purified mRNA is then capped and tailed, and subjected to a second precipitation and purification.

In some embodiments, a suitable mRNA sequence is an mRNA sequence encoding a protein or a peptide. In some embodiments, a suitable mRNA sequence is codon optimized for efficient expression in human cells. Codon optimization typically includes modifying a naturally-occurring or wild-type nucleic acid sequence encoding a peptide, polypeptide or protein to achieve the highest possible G/C content, to adjust codon usage to avoid rare or rate-limiting codons, to remove destabilizing nucleic acid sequences or motifs and/or to eliminate pause sites or terminator sequences without altering the amino acid sequence of the mRNA encoded peptide, polypeptide or protein. In some embodiments, a suitable mRNA sequence is naturally-occurring or a wild-type sequence. In some embodiments, a suitable mRNA sequence encodes a protein or a peptide that contains one or mutations in amino acid sequence.

The method according to the present invention can be used to prepare mRNAs of a variety of lengths. In some embodiments, the present invention may be used to prepare in vitro synthesized mRNA of or greater than about 0.5 kb, 1 kb, 1.5 kb, 2 kb, 2.5 kb, 3 kb, 3.5 kb, 4 kb, 4.5 kb, 5 kb 6 kb, 7 kb, 8 kb, 9 kb, 10 kb, 11 kb, 12 kb, 13 kb, 14 kb, 15 kb, 20 kb, 30 kb, 40 kb, or 50 kb in length. In some embodiments, the present invention may be used to deliver in vitro synthesized mRNA ranging from about 1-20 kb, about 1-15 kb, about 1-10 kb, about 5-20 kb, about 5-15 kb, about 5-12 kb, about 5-10 kb, about 8-20 kb, or about 8-50 kb in length. Accordingly, the method of the present invention can be used to prepare mRNAs of any gene of interest.

IVT Reaction

IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNase inhibitor. The exact conditions will vary according to the specific application. A suitable DNA template typically has a promoter, for example a T3, T7 or SP6 promoter, for in vitro transcription, followed by desired nucleotide sequence for desired mRNA and a termination signal. In some embodiments, the mRNA generated is codon optimized.

In some embodiments, an exemplary IVT reaction mixture contains linearized double stranded DNA template with an SP6 polymerase-specific promoter, SP6 RNA polymerase, RNase inhibitor, pyrophosphatase, 29 mM NTPs, 10 mM DTT and a reaction buffer (when at 10× is 800 mM HEPES, 20 mM spermidine, 250 mM MgCl₂, pH 7.7) and quantity sufficient (QS) to a desired reaction volume with RNase-free water; this reaction mixture is then incubated at 37° C. for 60 minutes. The polymerase reaction is then quenched by addition of DNase I and a DNase I buffer (when at 10× is 100 mM Tris-HCl, 5 mM MgCl₂ and 25 mM CaCl₂, pH 7.6) to facilitate digestion of the double-stranded DNA template in preparation for purification. This embodiment has been shown to be sufficient to produce 100 grams of mRNA

Other IVT methods are available in the art and may be used to practice the present invention.

Post-Synthesis Processing

Typically, a 5′ cap and/or a 3′ tail may be added after the synthesis. The presence of the cap is important in providing resistance to nucleases found in most eukaryotic cells. The presence of a “tail” serves to protect the mRNA from exonuclease degradation.

Capping and Tailing (C/T) Reactions

Typically, in eukaryotic organisms, mRNA processing comprises the addition of a “cap” on the N-terminal (5′) end, and a “tail” on the C-terminal (3′) end. A typical cap is a 7-methylguanosine cap, which is a guanosine that is linked through a 5′-5′-triphosphate bond to the first transcribed nucleotide. In some embodiment, the in vitro transcribed mRNA is modified enzymatically by the addition of a 5′ N⁷-methylguanylate Cap 0 structure using guanylate transferase and the addition of a methyl group at the 2′ 0 position of the penultimate nucleotide resulting in a Cap 1 structure using 2′ O-methyltransferase as described by Fechter, P.; Brownlee, G. G. “Recognition of mRNA cap structures by viral and cellular proteins” J. Gen. Virology 2005, 86, 1239-1249. For capping as part of the IVT reaction, a cap analog can be incorporated as the first “base” in the nascent RNA strand. The cap analog may be Cap 0, Cap1, Cap 2, ^{m6}Aₘ, or unnatural caps.

In some embodiments, a 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′-5′ triphosphate linkage; and the 7-nitrogen of guanine is then methylated by a methyltransferase. Examples of cap structures include, but are not limited to, m7G(5′)ppp (5′)G, G(5′)ppp(5′)A and G(5′)ppp(5′)G. Briefly, purified IVT mRNA is typically mixed with GTP, S-adenosyl methionine, RNase inhibitor, 2′-Omethyl transferase, guanylyl transferase, in the presence of a reaction buffer comprising Tris-HCl, MgCl₂, and RNase-free H₂O; then incubated at 37° C. Conventional capping reaction buffer comprises 50 mM Tris-HCl pH 8.0 and 1.25 mM MgCl₂.

In some embodiments, following addition of the Cap 1 structure, a poly-adenylate tail is added to the 3′ end of the in vitro transcribed mRNA enzymatically using poly-A polymerase. The tail is typically a polyadenylation event whereby a polyadenylyl moiety is added to the 3′ end of the mRNA molecule. In some embodiments, following the incubation for capping reaction, a tailing reaction is initiated by adding tailing buffer comprising Tris-HCl, NaCl, MgCl₂, ATP, poly A polymerase and RNase-free H₂O. The reaction is quenched by addition of EDTA. Conventional tailing reaction buffer comprises 50 mM Tris-HCl pH 8.0 and 1.25 mM MgCl₂.

In some embodiments, the pH of the optimized reaction buffer of the present invention is between about 6.0 and 8.0. In some embodiments, the pH of the reaction buffer is between about 6.5 and 8.0. In some embodiments, the pH of the reaction buffer is between about 7.0 and 7.8. In some embodiments, the pH of the reaction buffer is between about 7.2 and 7.7. In some embodiments, the pH of the reaction buffer is between about 7.4 and 7.6. In some embodiments, the pH of the reaction buffer is about 7.0. In some embodiments, the pH of the reaction buffer is about 7.2. In some embodiments, the pH of the reaction buffer is about 7.3. In some embodiments, the pH of the reaction buffer is about 7.4. In some embodiments, the pH of the reaction buffer is about 7.5. In some embodiments, the pH of the reaction buffer is about 7.6. In some embodiments, the pH of the reaction buffer is about 7.7. In some embodiments, the pH of the reaction buffer is about 7.8. In some embodiments, the pH of the reaction buffer is about 8.0.

In some embodiments, the MgCl₂ in the optimized reaction buffer of the present invention has a concentration of about between 0.10 mM and 1.25. In some embodiments, the MgCl₂ in the reaction buffer has a concentration of about between 0.75 mM and 1.25 mM. In some embodiments, the MgCl₂ in the reaction buffer has a concentration of about between 0.50 mM and 1.0 mM. In some embodiments, the MgCl₂ in the reaction buffer has a concentration of about between 0.75 mM and 1.0 mM. In some embodiments, the MgCl₂ in the reaction buffer has a concentration of 0.25 mM. In some embodiments, the MgCl₂ in the reaction buffer has a concentration of 0.5 mM. In some embodiments, the MgCl₂ in the reaction buffer has a concentration of 0.7 mM. In some embodiments, the MgCl₂ in the reaction buffer has a concentration of 0.75 mM. In some embodiments, the MgCl₂ in the reaction buffer has a concentration of 0.8 mM. In some embodiments, the MgCl₂ in the reaction buffer has a concentration of 0.9 mM. In some embodiments, the MgCl₂ in the reaction buffer has a concentration of 1.0 mM. In some embodiments, the MgCl₂ in the reaction buffer has a concentration of 1.10 mM. In some embodiments, the MgCl₂ in the reaction buffer has a concentration of 1.20 mM.

mRNA Purification

In some embodiments, mRNAs prior and post capping and tailing reaction may be further purified. Various methods may be used to purify mRNA synthesized according to methods known in the art. For example, purification of mRNA can be performed using centrifugation, filtration and/or chromatographic methods. In some embodiments, the synthesized mRNA is purified by ethanol precipitation or filtration or chromatography, or gel purification or any other suitable means. In some embodiments, the mRNA is purified by HPLC. In some embodiments, the mRNA is extracted in a standard phenol:chloroform:isoamyl alcohol solution, well known to one of skill in the art. In some embodiments, the mRNA is purified using Tangential Flow Filtration. Suitable purification methods include those described in published U.S. Application No. US 2016/0040154, published U.S. Application No. US 2015/0376220, published U.S. Application No. US 2018/0251755, published U.S. Application No. US 2018/0251754, U.S. Provisional Application No. 62/757,612 filed on Nov. 8, 2018, and U.S. Provisional Application No. 62/891,781 filed on Aug. 26, 2019, all of which are incorporated by reference herein and may be used to practice the present invention.

In some embodiments, the mRNA is purified before capping and tailing. In some embodiments, the mRNA is purified after capping and tailing. In some embodiments, the mRNA is purified both before and after capping and tailing. In general, a purification step as described herein may be performed after each step of mRNA synthesis, optionally along with other purification processes, such as dialysis.

In some embodiments, the mRNA is purified either before or after or both before and after capping and tailing, by centrifugation.

In some embodiments, the mRNA is purified either before or after or both before and after capping and tailing, by filtration.

In some embodiments, the mRNA is purified either before or after or both before and after capping and tailing, by Tangential Flow Filtration (TFF).

In some embodiments, the mRNA is purified either before or after or both before and after capping and tailing by chromatography.

Precipitation of mRNA

mRNA in an impure preparation, such as an in vitro synthesis reaction mixture may be precipitated using a buffer and suitable conditions as described in U.S. Provisional Application No. 62/757,612 filed on Nov. 8, 2018, or in U.S. Provisional Application No. 62/891,781 filed on Aug. 26, 2019, and may be used to practice the present invention followed by various methods of purification known in the art. As used herein, the term “precipitation” (or any grammatical equivalent thereof) refers to the formation of an insoluble substance (e.g., solid) in a solution. When used in connection with mRNA, the term “precipitation” refers to the formation of insoluble or solid form of mRNA in a liquid.

Typically, mRNA precipitation involves a denaturing condition. As used herein, the term “denaturing condition” refers to any chemical or physical condition that can cause disruption of native confirmation of mRNA. Since the native conformation of a molecule is usually the most water soluble, disrupting the secondary and tertiary structures of a molecule may cause changes in solubility and may result in precipitation of mRNA from solution.

For example, a suitable method of precipitating mRNA from an impure preparation involves treating the impure preparation with a denaturing reagent such that the mRNA precipitates. Exemplary denaturing reagents suitable for the invention include, but are not limited to, lithium chloride, sodium chloride, potassium chloride, guanidinium chloride, guanidinium thiocyanate, guanidinium isothiocyanate, ammonium acetate and combinations thereof. Suitable reagent may be provided in a solid form or in a solution.

In some embodiments, a guanidinium salt is used in a denaturation buffer for precipitating mRNA. As non-limiting examples, guanidinium salts may include guanidinium chloride, guanidinium thiocyanate, or guanidinium isothiocyanate. Guanidinium thiocyanate, also termed as guanidine thiocyanate may be used to precipitate mRNA. The present invention is based on the surprising discovery that in an mRNA precipitating buffer comprising guanidinium salts, such as Guanidinium thiocyanate can be used at a concentration higher than is typically used for denaturing reactions, resulting in mRNA that is substantially free of protein contaminants. In some embodiments, a solution suitable for mRNA precipitation contains guanidine thiocyanate at a concentration greater than 4 M.

In some embodiments, a buffer comprising a denaturing reagent suitable for mRNA precipitation comprises greater than 4 M guanidine thiocyanate. In some embodiments, a buffer comprising a denaturing reagent suitable for mRNA precipitation comprises about 5 M GSCN. In some embodiments, a buffer comprising a denaturing reagent suitable for mRNA precipitation comprises about 5.5 M GSCN. In some embodiments, a buffer comprising a denaturing reagent suitable for mRNA precipitation comprises about 6 M GSCN. In some embodiments, a buffer comprising a denaturing reagent suitable for mRNA precipitation comprises about 6.5 M GSCN. In some embodiments, a buffer comprising a denaturing reagent suitable for mRNA precipitation comprises about 7 M GSCN. In some embodiments, a buffer comprising a denaturing reagent suitable for mRNA precipitation comprises about 7.5 M GSCN. In some embodiments, a buffer comprising a denaturing reagent suitable for mRNA precipitation comprises about 8 M GSCN. In some embodiments, a buffer comprising a denaturing reagent suitable for mRNA precipitation comprises about 8.5 M GSCN. In some embodiments, a buffer comprising a denaturing reagent suitable for mRNA precipitation comprises about 9 M GSCN. In some embodiments, a buffer comprising a denaturing reagent suitable for mRNA precipitation comprises about 10 M GSCN. In some embodiments, a buffer comprising a denaturing reagent suitable for mRNA precipitation comprises greater than 10 M GSCN.

In addition to denaturing reagent, a suitable solution for mRNA precipitation may include additional salt, surfactant and/or buffering agent. For example, a suitable solution may further include sodium lauryl sarcosyl and/or sodium citrate. In some embodiments, a buffer suitable for mRNA precipitation comprises about 5 mM sodium citrate. In some embodiments, a buffer suitable for mRNA precipitation comprises about 10 mM sodium citrate. In some embodiments, a buffer suitable for mRNA precipitation comprises about 20 mM sodium citrate. In some embodiments, a buffer suitable for mRNA precipitation comprises about 25 mM sodium citrate. In some embodiments, a buffer suitable for mRNA precipitation comprises about 30 mM sodium citrate. In some embodiments, a buffer suitable for mRNA precipitation comprises about 50 mM sodium citrate.

In some embodiments, a buffer suitable for mRNA precipitation comprises a surfactant, such as N-Lauryl Sarcosine (Sarcosyl). In some embodiments, a buffer suitable for mRNA precipitation comprises about 0.01% N-Lauryl Sarcosine. In some embodiments, a buffer suitable for mRNA precipitation comprises about 0.05% N-Lauryl Sarcosine. In some embodiments, a buffer suitable for mRNA precipitation comprises about 0.1% N-Lauryl Sarcosine. In some embodiments, a buffer suitable for mRNA precipitation comprises about 0.5% N-Lauryl Sarcosine. In some embodiments, a buffer suitable for mRNA precipitation comprises 1% N-Lauryl Sarcosine. In some embodiments, a buffer suitable for mRNA precipitation comprises about 1.5% N-Lauryl Sarcosine. In some embodiments, a buffer suitable for mRNA precipitation comprises about 2%, about 2.5% or about 5% N-Lauryl Sarcosine.

In some embodiments, a suitable solution for mRNA precipitation comprises a reducing agent. In some embodiments, the reducing agent is selected from dithiothreitol (DTT), beta-mercaptoethanol (b-ME), Tris(2-carboxyethyl)phosphine (TCEP), Tris(3-hydroxypropyl)phosphine (THPP), dithioerythritol (DTE) and dithiobutylamine (DTBA). In some embodiments, the reducing agent is dithiothreitol (DTT).

In some embodiments, DTT is present at a final concentration that is greater than 1 mM and up to about 200 mM. In some embodiments, DTT is present at a final concentration between 2.5 mM and 100 mM. In some embodiments, DTT is present at a final concentration between 5 mM and 50 mM.

In some embodiments, DTT is present at a final concentration of 1 mM or greater. In some embodiments, DTT is present at a final concentration of 2 mM or greater. In some embodiments, DTT is present at a final concentration of 3 mM or greater. In some embodiments, DTT is present at a final concentration of 4 mM or greater. In some embodiments, DTT is present at a final concentration of 5 mM or greater. In some embodiments, DTT is present at a final concentration of 6 mM or greater. In some embodiments, DTT is present at a final concentration of 7 mM or greater. In some embodiments, DTT is present at a final concentration of 8 mM or greater. In some embodiments, DTT is present at a final concentration of 9 mM or greater. In some embodiments, DTT is present at a final concentration of 10 mM or greater. In some embodiments, DTT is present at a final concentration of 11 mM or greater. In some embodiments, DTT is present at a final concentration of 12 mM or greater. In some embodiments, DTT is present at a final concentration of 13 mM or greater. In some embodiments, DTT is present at a final concentration of 14 mM or greater. In some embodiments, DTT is present at a final concentration of 15 mM or greater. In some embodiments, DTT is present at a final concentration of 16 mM or greater. In some embodiments, DTT is present at a final concentration of 17 mM or greater. In some embodiments, DTT is present at a final concentration of 18 mM or greater. In some embodiments, DTT is present at a final concentration of 19 mM or greater. In some embodiments, DTT is present at a final concentration of about 20 mM.

In some embodiments, the denaturing buffer comprises 2 M GSCN or greater, and DTT. In some embodiments, the denaturing buffer comprises 3 M GSCN or greater, and DTT. In some embodiments, the denaturing buffer comprises 4 M GSCN or greater, and DTT. In some embodiments, the denaturing buffer comprises about 5 M GSCN or greater, and DTT. In some embodiments, the denaturing buffer comprises about 6 M GSCN or greater, and DTT. In some embodiments, the denaturing buffer comprises about 7 M GSCN or greater, and DTT. In some embodiments, the denaturing buffer comprises about 8 M GSCN or greater, and DTT. In some embodiments, the denaturing buffer comprises about 9 M GSCN or greater, and DTT.

In some embodiments, the denaturing buffer comprises 1 mM DTT or greater and GSCN concentration of about 5 M. In some embodiments, the denaturing buffer comprises 2 mM DTT or greater and GSCN concentration of about 5 M. In some embodiments, the denaturing buffer comprises 3 mM DTT or greater and GSCN concentration of about 5 M. In some embodiments, the denaturing buffer comprises 4 mM DTT or greater and GSCN concentration of about 5 M. In some embodiments, the denaturing buffer comprises 5 mM DTT or greater and GSCN concentration of about 5 M. In some embodiments, the denaturing buffer comprises 6 mM DTT or greater and GSCN concentration of about 5 M. In some embodiments, the denaturing buffer comprises 7 mM DTT or greater and GSCN concentration of about 5 M. In some embodiments, the denaturing buffer comprises 8 mM DTT or greater and GSCN concentration of about 5 M. In some embodiments, the denaturing buffer comprises 9 mM DTT or greater and GSCN concentration of about 5 M. In some embodiments, the denaturing buffer comprises 10 mM DTT or greater and GSCN concentration of about 5 M.

Protein denaturation may occur even at a low concentration of the denaturation reagent, when in the presence or absence of the reducing agent. The combination of a high concentration of GSCN and a high concentration of DTT in a denaturing solution for precipitating an mRNA containing impurities yields mRNA which is pure and substantially free of protein contaminants. mRNA precipitated in the buffer can be processed through a filter. In some embodiments, the eluent after a single precipitation followed by filtration using the buffer comprising about 5 M GSCN and about 10 mM DTT is of high quality and purity with no detectable proteins impurities. Additionally, the method is reproducible at wide range of the amount of mRNA processed, in the scales involving about 1 gram, or about 10 grams, or about 100 grams, or about 500 grams, or about 1000 grams of mRNA and more, without causing hindrance in flow of fluids through a filter.

In some embodiments, the buffer for the precipitating step further comprises an alcohol. In some embodiments, the precipitating is performed under conditions where the mRNA, denaturing buffer (comprising GSCN and reducing agent, e.g. DTT) and alcohol are present in a volumetric ratio of 1:(5):(3). In some embodiments, the precipitating is performed under conditions where the mRNA, denaturing buffer and alcohol are present in a volumetric ratio of 1:(3.5):(2.1). In some embodiments, the precipitating is performed under conditions where the mRNA, denaturing buffer and alcohol are present in a volumetric ratio of 1:(4):(2). In some embodiments, the precipitating is performed under conditions where the mRNA, denaturing buffer and alcohol are present in a volumetric ratio of 1:(2.8):(1.9). In some embodiments, the precipitating is performed under conditions where the mRNA, denaturing buffer and alcohol are present in the volumetric ratio of 1:(2.3):(1.7). In some embodiments, the precipitating is performed under conditions where the mRNA, denaturing buffer and alcohol are present in the volumetric ratio of 1:(2.1):(1.5).

In some embodiments, it is desirable to incubate the impure preparation with one or more denaturing reagents described herein for a period of time at a desired temperature that permits precipitation of substantial amount of mRNA. For example, the mixture of an impure preparation and a denaturing agent may be incubated at room temperature or ambient temperature for a period of time. In some embodiments, a suitable incubation time is a period of or greater than about 2, 3, 4, 5, 6, 7, 8, 9, 10, 15, 20, 25, 30, 40, 50, or 60 minutes. In some embodiments, a suitable incubation time is a period of or less than about 60, 55, 50, 45, 40, 35, 30, 25, 20, 15, 10, 9, 8, 7, 6, or 5 minutes. In some embodiments, the mixture is incubated for about 5 minutes at room temperature. Typically, “room temperature” or “ambient temperature” refers to a temperature with the range of about 20-25° C., for example, about 20° C., 21° C., 22° C., 23° C., 24° C., or 25° C. In some embodiments, the mixture of an impure preparation and a denaturing agent may also be incubated above room temperature (e.g., about 30-37° C. or in particular, at about 30° C., 31° C., 32° C., 33° C., 34° C., 35° C., 36° C., or 37° C.) or below room temperature (e.g., about 15-20° C., or in particular, at about 15° C., 16° C., 17° C., 18° C., 19° C., or 20° C.). The incubation period may be adjusted based on the incubation temperature. Typically, a higher incubation temperature requires shorter incubation time.

Alternatively or additionally, a solvent may be used to facilitate mRNA precipitation. Suitable exemplary solvent includes, but is not limited to, isopropyl alcohol, acetone, methyl ethyl ketone, methyl isobutyl ketone, ethanol, methanol, denatonium, and combinations thereof. For example, a solvent (e.g., absolute ethanol) may be added to an impure preparation together with a denaturing reagent or after the addition of a denaturing reagent and the incubation as described herein, to further enhance and/or expedite mRNA precipitation. Typically, after the addition of a suitable solvent (e.g., absolute ethanol), the mixture may be incubated at room temperature for another period of time. Typically, a suitable period of incubation time is or greater than about 2, 3, 4, 5, 6, 7, 8, 9, 10, 15, 20, 25, 30, 40, 50, or 60 minutes. In some embodiments, a suitable period of incubation is a period of or less than about 60, 55, 50, 45, 40, 35, 30, 25, 20, 15, 10, 9, 8, 7, 6, or 5 minutes. Typically, the mixture is incubated at room temperature for about 5 minutes. Temperature above or below room may be used with proper adjustment of incubation time. Alternatively, incubation could occur at 4° C. or −20° C. for precipitation.

In some embodiments, precipitating the mRNA in a suspension comprises one or more amphiphilic polymers. In some embodiments, the precipitating the mRNA in a suspension comprises a PEG polymer. Various kinds of PEG polymers are recognized in the art, some of which have distinct geometrical configurations. PEG polymers include, for example, PEG polymers having linear, branched, Y-shaped, or multi-arm configuration. In some embodiments, the PEG is in a suspension comprising one or more PEG of distinct geometrical configurations. In some embodiments, precipitating mRNA can be achieved using PEG-6000 to precipitate the mRNA. In some embodiments, precipitating mRNA can be achieved using PEG-400 to precipitate the mRNA. In some embodiments, precipitating mRNA can be achieved using triethylene glycol (TEG) to precipitate the mRNA. In some embodiments, precipitating mRNA can be achieved using triethylene glycol monomethyl ether (MTEG) to precipitate the mRNA. In some embodiments, precipitating mRNA can be achieved using tert-butyl-TEG-O-propionate to precipitate the mRNA. In some embodiments, precipitating mRNA can be achieved using TEG-dimethacrylate to precipitate the mRNA. In some embodiments, precipitating mRNA can be achieved using TEG-dimethyl ether to precipitate the mRNA. In some embodiments, precipitating mRNA can be achieved using TEG-divinyl ether to precipitate the mRNA. In some embodiments, precipitating mRNA can be achieved using TEG-monobutyl ether to precipitate the mRNA. In some embodiments, precipitating mRNA can be achieved using TEG-methyl ether methacrylate to precipitate the mRNA. In some embodiments, precipitating mRNA can be achieved using TEG-monodecyl ether to precipitate the mRNA. In some embodiments, precipitating mRNA can be achieved using TEG-dibenzoate to precipitate the mRNA. Any one of these PEG or TEG based reagents can be used in combination with guanidinium thiocyanate to precipitate the mRNA.

Many amphiphilic polymers are known in the art. In some embodiments, amphiphilic polymer include pluronics, polyvinyl pyrrolidone, polyvinyl alcohol, polyethylene glycol (PEG), or combinations thereof. In some embodiments, the amphiphilic polymer is selected from one or more of the following: PEG triethylene glycol, tetraethylene glycol, PEG 200, PEG 300, PEG 400, PEG 600, PEG 1,000, PEG 1,500, PEG 2,000, PEG 3,000, PEG 3,350, PEG 4,000, PEG 6,000, PEG 8,000, PEG 10,000, PEG 20,000, PEG 35,000, and PEG 40,000, or combination thereof. In some embodiments, the amphiphilic polymer comprises a mixture of two or more kinds of molecular weight PEG polymers are used. For example, in some embodiments, two, three, four, five, six, seven, eight, nine, ten, eleven, or twelve molecular weight PEG polymers comprise the amphiphilic polymer. Accordingly, in some embodiments, the PEG solution comprises a mixture of one or more PEG polymers. In some embodiments, the mixture of PEG polymers comprises polymers having distinct molecular weights.

In some embodiments, precipitating the mRNA in a suspension comprises a PEG polymer, wherein the PEG polymer comprises a PEG-modified lipid. In some embodiments, the PEG-modified lipid is 1,2-dimyristoyl-sn-glycerol, methoxypolyethylene glycol (DMG-PEG-2K). In some embodiments, the PEG modified lipid is a DOPA-PEG conjugate. In some embodiments, the PEG-modified lipid is a poloxamer-PEG conjugate. In some embodiments, the PEG-modified lipid comprises DOTAP. In some embodiments, the PEG-modified lipid comprises cholesterol.

In some embodiments, the mRNA is precipitated in suspension comprising an amphiphilic polymer. In some embodiments, the mRNA is precipitated in a suspension comprising any of the aforementioned PEG reagents. In some embodiments, PEG is in the suspension at about 10% to about 100% weight/volume concentration. For example, in some embodiments, PEG is present in the suspension at about 5%, 10%, 15%, 20%, 25%, 30%, 35%, 40%, 45%, 50%, 55%, 60%, 65%, 70%, 75%, 80%, 85%, 90%, 95%, 100% weight/volume concentration, and any values there between. In some embodiments, PEG is present in the suspension at about 5% weight/volume concentration. In some embodiments, PEG is present in the suspension at about 6% weight/volume concentration. In some embodiments, PEG is present in the suspension at about 7% weight/volume concentration. In some embodiments, PEG is present in the suspension at about 8% weight/volume concentration. In some embodiments, PEG is present in the suspension at about 9% weight/volume concentration. In some embodiments, PEG is present in the suspension at about 10% weight/volume concentration. In some embodiments, PEG is present in the suspension at about 12% weight/volume concentration. In some embodiments, PEG is present in the suspension at about 15% weight/volume. In some embodiments, PEG is present in the suspension at about 18% weight/volume. In some embodiments, PEG is present in the suspension at about 20% weight/volume concentration. In some embodiments, PEG is present in the suspension at about 25% weight/volume concentration. In some embodiments, PEG is present in the suspension at about 30% weight/volume concentration. In some embodiments, PEG is present in the suspension at about 35% weight/volume concentration. In some embodiments, PEG is present in the suspension at about 40% weight/volume concentration. In some embodiments, PEG is present in the suspension at about 45% weight/volume concentration. In some embodiments, PEG is present in the suspension at about 50% weight/volume concentration. In some embodiments, PEG is present in the suspension at about 55% weight/volume concentration. In some embodiments, PEG is present in the suspension at about 60% weight/volume concentration. In some embodiments, PEG is present in the suspension at about 65% weight/volume concentration. In some embodiments, PEG is present in the suspension at about 70% weight/volume concentration. In some embodiments, PEG is present in the suspension at about 75% weight/volume concentration. In some embodiments, PEG is present in the suspension at about 80% weight/volume concentration. In some embodiments, PEG is present in the suspension at about 85% weight/volume concentration. In some embodiments, PEG is present in the suspension at about 90% weight/volume concentration. In some embodiments, PEG is present in the suspension at about 95% weight/volume concentration. In some embodiments, PEG is present in the suspension at about 100% weight/volume concentration.

In some embodiments, precipitating the mRNA in a suspension comprises a volume:volume ratio of PEG to total mRNA suspension volume of about 0.1 to about 5.0. For example, in some embodiments, PEG is present in the mRNA suspension at a volume:volume ratio of about 0.1, 0.2, 0.3, 0.4, 0.5, 0.6, 0.7, 0.8, 0.9, 1.0, 1.25, 1.5, 1.75, 2.0, 2.25, 2.5, 2.75, 3.0, 3.25, 3.5, 3.75, 4.0, 4.25, 4.5, 4.75, 5.0. Accordingly, in some embodiments, PEG is present in the mRNA suspension at a volume:volume ratio of about 0.1. In some embodiments, PEG is present in the mRNA suspension at a volume:volume ratio of about 0.2. In some embodiments, PEG is present in the mRNA suspension at a volume:volume ratio of about 0.3. In some embodiments, PEG is present in the mRNA suspension at a volume:volume ratio of about 0.4. In some embodiments, PEG is present in the mRNA suspension at a volume:volume ratio of about 0.5. In some embodiments, PEG is present in the mRNA suspension at a volume:volume ratio of about 0.6. In some embodiments, PEG is present in the mRNA suspension at a volume:volume ratio of about 0.7. In some embodiments, PEG is present in the mRNA suspension at a volume:volume ratio of about 0.8. In some embodiments, PEG is present in the mRNA suspension at a volume:volume ratio of about 0.9. In some embodiments, PEG is present in the mRNA suspension at a volume:volume ratio of about 1.0. In some embodiments, PEG is present in the mRNA suspension at a volume:volume ratio of about 1.25. In some embodiments, PEG is present in the mRNA suspension at a volume:volume ratio of about 1.5. In some embodiments, PEG is present in the mRNA suspension at a volume:volume ratio of about 1.75. In some embodiments, PEG is present in the mRNA suspension at a volume:volume ratio of about 2.0. In some embodiments, PEG is present in the mRNA suspension at a volume:volume ratio of about 2.25. In some embodiments, PEG is present in the mRNA suspension at a volume:volume ratio of about 2.5. In some embodiments, PEG is present in the mRNA suspension at a volume:volume ratio of about 2.75. In some embodiments, PEG is present in the mRNA suspension at a volume:volume ratio of about 3.0. In some embodiments, PEG is present in the mRNA suspension at a volume:volume ratio of about 3.25. In some embodiments, PEG is present in the mRNA suspension at a volume:volume ratio of about 3.5. In some embodiments, PEG is present in the mRNA suspension at a volume:volume ratio of about 3.75. In some embodiments, PEG is present in the mRNA suspension at a volume:volume ratio of about 4.0. In some embodiments, PEG is present in the mRNA suspension at a volume:volume ratio of about 4.25. In some embodiments, PEG is present in the mRNA suspension at a volume:volume ratio of about 4.50. In some embodiments, PEG is present in the mRNA suspension at a volume:volume ratio of about 4.75. In some embodiments, PEG is present in the mRNA suspension at a volume:volume ratio of about 5.0.

In some embodiments, a reaction volume for mRNA precipitation comprises GSCN and PEG.

In some embodiments, the method of purifying mRNA is alcohol free.

In some embodiments, a non-aqueous solvent (e.g., alcohol) is added to precipitate mRNA. In some embodiments, a solvent may be isopropyl alcohol, acetone, methyl ethyl ketone, methyl isobutyl ketone, ethanol, methanol, denatonium, and combinations thereof. In embodiments, a solvent is an alcohol solvent (e.g., methanol, ethanol, or isopropanol). In embodiments, a solvent is a ketone solvent (e.g., acetone, methyl ethyl ketone, or methyl isobutyl ketone). In some embodiments, a non-aqueous solvent is mixed with the amphiphilic solution.

In some embodiments, an aqueous solution is added to precipitate mRNA. In some embodiments, the aqueous solution comprises a polymer. In some embodiments, the aqueous solution comprises a PEG polymer.

In some embodiments, the method further includes a step of adding one or more agents that denature proteins (e.g., RNA polymerase and DNase I, which is added after transcription to remove DNA templates) and/or keep proteins soluble in an aqueous medium. In some embodiments, the one or more agents that denature proteins and/or keep proteins soluble in an aqueous medium is a salt, e.g., a chaotropic salt.

In some embodiments, a precipitating step comprises the use of a chaotropic salt (e.g., guanidine thiocyanate) and/or an amphiphilic polymer (e.g., polyethylene glycol or an aqueous solution of polyethylene glycol) and/or an alcohol solvent (e.g., absolute ethanol or an aqueous solution of alcohol such as an aqueous ethanol solution). Accordingly, in some embodiments, the precipitating step comprises the use of a chaotropic salt and an amphiphilic polymer, such as GSCN and PEG, respectively.

In some embodiments, agents that promote precipitation of mRNA include a denaturing agent or result from denaturing conditions. As used herein, the term “denaturing condition” refers to any chemical or physical conditions that can cause denaturation. Exemplary denaturing conditions include, but are not limited to, use of chemical reagents, high temperatures, extreme pH, etc. In some embodiments, a denaturing condition is achieved through adding one or more denaturing agents to an impure preparation containing mRNA to be purified. In some embodiments, a denaturing agent suitable for the present invention is a protein and/or DNA denaturing agent. In some embodiments, a denaturing agent may be: 1) an enzyme (such as a serine proteinase or a DNase), 2) an acid, 3) a solvent, 4) a cross-linking agent, 5) a chaotropic agent, 6) a reducing agent, and/or 7) high ionic strength via high salt concentrations. In some embodiments, a particular agent may fall into more than one of these categories.

Nucleotides

In some embodiments, an mRNA comprises or consists of naturally-occurring nucleosides (or unmodified nucleosides; i.e., adenosine, guanosine, cytidine, and uridine). In some embodiments an mRNA comprises one or more modified nucleosides (e.g. adenosine analog, guanosine analog, cytidine analog, or uridine analog). In some embodiments, an mRNA comprises both unmodified and modified nucleosides. In some embodiments, the one or more modified nucleosides is a nucleoside analog. In some embodiments, the one or more modified nucleosides comprises at least one modification selected from a modified sugar, and a modified nucleobase. In some embodiments, the mRNA comprises one or more modified internucleoside linkages.

In some embodiments, the one or more modified nucleosides is a nucleoside analog, for example one of 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, pseudouridine (e.g., N-1-methyl-pseudouridine), 2-thiouridine, and 2-thiocytidine. See, e.g., U.S. Pat. No. 8,278,036 or WO 2011/012316 for a discussion of 5-methyl-cytidine, pseudouridine, and 2-thio-uridine and their incorporation into mRNA. In some embodiments, the mRNA may be RNA wherein 25% of U residues are 2-thio-uridine and 25% of C residues are 5-methylcytidine. Teachings for the use of such modified RNA are disclosed in US Patent Publication US 2012/0195936 and international publication WO 2011/012316, both of which are hereby incorporated by reference in their entirety. In some embodiments, the presence of one or more nucleoside analogs may render an mRNA more stable and/or less immunogenic than a control mRNA with the same sequence but containing only naturally-occurring nucleosides.

In some embodiments, the one or more modified nucleosides comprises a modified nucleobase, for example a chemically modified base, a biologically modified base (e.g., a methylated base); or an intercalated base. In some embodiments, the one or more modified nucleosides comprises a modified nucleobase selected from a modified purine (adenine (A), guanine (G)) or a modified pyrimidine (thymine (T), cytosine (C), uracil (U)), such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, beta-D-mannosyl-queosine, wybutoxosine, and phosphoramidates, phosphorothioates, peptide nucleotides, methylphosphonates, 7-deazaguanosine, 5-methylcytosine, inosine, isocytosine, pseudoisocytosine, 5-bromouracil, 5-propynyluracil, 6-aminopurine, 2-aminopurine, diaminopurine and 2-chloro-6-aminopurine cytosine. The preparation of such modified nucleobases is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosures of which are incorporated by reference in their entirety.

In some embodiments, the mRNA comprises one or more modified internucleoside linkages. For example, one or more of the modified nucleotides used to produce the mRNA of the invention may comprise a modified phosphate group. Therefore, in the mRNA, one or more phosphodiester linkages is substituted with another anionic, cationic or neutral group. For example, in some embodiments the one or more modified nucleotides comprises a modified phosphate group selected from methylphosphonates, methylphosphoramidates, phosphoramidates, phosphorothioates (e.g., cytidine 5′-O-(1-thiophosphate)), boranophosphates, and positively charged guanidinium groups. In some embodiments the one or more modified internucleoside linkages is a phosphorothioate linkage. In some embodiments the one or more modified internucleoside linkages is a 5′-N-phosphoramidite linkage.

In some embodiments, the one or more modified nucleosides comprises a modified sugar. In some embodiments the one or more modified nucleosides comprises a modification to the furanose ring. In some embodiments the one or more modified nucleosides comprises a modified sugar selected from 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate). In some embodiments the one or more modified nucleosides comprises a modified sugar selected from a 2′-O-alkyl modification or a locked nucleic acid (LNA)). In some embodiments, where the sugar modification is a 2′-O-alkyl modification, such modification may include, but are not limited to a 2′-deoxy-2′-fluoro modification, a 2′-O-methyl modification, a 2′-O-methoxyethyl modification and a 2′-deoxy modification. In some embodiments the one or more modified nucleosides comprises a modified sugar selected from 2′-fluororibose, ribose, 2′-deoxyribose, arabinose, and hexose.

In some embodiments, any of these modifications may be present in 0-100% of the nucleotides—for example, more than 0%, 1%, 10%, 25%, 50%, 75%, 85%, 90%, 95%, or 100% of the constituent nucleotides individually or in combination.

In some embodiments, the RNAs may be complexed or hybridized with additional polynucleotides and/or peptide polynucleotides (PNA).

Purified mRNA Product

The method of capping and tailing an in vitro transcribed purified mRNA according to the present invention results in high RNA integrity and capping tailing efficiency. The purified capped and mRNA made according to the present invention is substantially free of contaminants comprising short abortive RNA species, long abortive RNA species, double-stranded RNA (dsRNA), residual plasmid DNA, residual in vitro transcription enzymes, residual solvent and/or residual salt.

The mRNA prepared according to the present invention encodes a protein or a peptide. The mRNAs prepared according to the present invention can encode any gene of interest, for example, as listed in published U.S. Application No. US 2017/0314041, which is incorporated herein by reference in its entirety. In some embodiments, the mRNA encodes Cystic Fibrosis Transmembrane Conductance Regulator (CFTR). In some embodiments, the mRNA encodes human phenylalanine hydroxylase (hPAH). In some embodiments, the mRNA encodes Ornithine transcarbamylase (OTC).

RNA Integrity

In some embodiments, assessing the quality of the mRNA includes assessment of mRNA integrity, capping and tailing efficiencies, 3′ tail length, purity, assessment of residual plasmid DNA, and assessment of residual solvent.

In some embodiments, mRNA products that are capped and tailed by present method are significantly more uniform and homogeneous enriched with full-length mRNA molecules as compared to the mRNA products that are capped and tailed by conventional methods which have a more heterogeneous profile with lower molecular weight pre-aborted transcripts present, when characterized by Glyoxal agarose gel electrophoresis or capillary electrophoresis after capping and tailing. Particularly, capping and tailing mRNAs in reaction conditions comprising Tris-HCl pH 7.5 buffer and 1.0 mM MgCl₂ resulted in RNA integrity of at least 70%. This unique and advantageous condition of capping and tailing reaction condition was not appreciated prior to the present invention and is truly unexpected especially because the optimized cap and tail condition is able to increase the RNA integrity by at least about 25%. Based on this unexpected discovery, the present inventors have successfully developed a large-scale production method to prepare mRNA molecules that have high RNA integrity suitable for mRNA therapeutics.

In various embodiments, a purified mRNA of the present invention maintains high degree of integrity. As used herein, the term “mRNA integrity” generally refers to the quality of mRNA after purification. mRNA integrity may be determined using methods well known in the art, for example, by RNA agarose gel electrophoresis. In some embodiments, mRNA integrity may be determined by banding patterns of RNA agarose gel electrophoresis. In some embodiments, a purified mRNA of the present invention shows little or no banding compared to reference band of RNA agarose gel electrophoresis.

In some embodiments, acceptable levels of mRNA integrity are assessed by agarose gel electrophoresis. The gels are analyzed to determine whether the banding pattern and apparent nucleotide length is consistent with an analytical reference standard. Additional methods to assess RNA integrity include, for example, assessment of the purified mRNA using capillary gel electrophoresis (CGE). In some embodiments, acceptable purity of the purified mRNA as determined by CGE is that the purified mRNA composition has no greater than about 55% long abortive/degraded species.

In some embodiments, at least 81%, 82%, 83%, 84%, 85%, 86%, 87%, 88%, 89%, 90%, 91%, 92%, 93%, 94%, 95%, 96%, 97%, 98%, 99%, 99.5%, or 99.9% of the mRNA products are full-length. In some embodiments, the mRNA products are substantially full-length.

In some embodiments, an mRNA composition includes less than 20%, 19%, 18%, 17%, 16%, 15%, 14%, 13%, 12%, 11%, 10%, 9%, 8%, 7%, 6%, 5%, 4%, 3%, 2%, 1%, 0.5%, or 0.1% of abortive transcripts. In some embodiments, an mRNA composition according to the present invention is substantially free of abortive transcripts.

In some embodiments, the full-length or abortive transcripts of mRNA are detected by gel electrophoresis (e.g., agarose gel electrophoresis) where the mRNA is denatured by Glyoxal before agarose gel electrophoresis (“Glyoxal agarose gel electrophoresis”). The mRNA synthesized according to the method of the invention contains undetectable amount of abortive transcripts on Glyoxal agarose gel electrophoresis.

In some embodiments, the full-length or abortive transcripts of mRNA are detected by capillary electrophoresis, e.g., capillary electrophoresis coupled with a fluorescence-based detection or capillary electrophoresis coupled with UV absorption spectroscopy detection. When detection is by capillary electrophoresis coupled with fluorescence based detection or by capillary electrophoresis coupled with UV absorption spectroscopy, the relative amount of full-length or abortive transcripts of synthesized mRNA is determined by the relative peak areas corresponding to the full-length or abortive transcripts.

Full-length or abortive transcripts of mRNA may be detected prior to capping and/or tailing the synthesized mRNA.

In some embodiments, the method further includes steps of capping and/or tailing the synthesized mRNA. The full-length or abortive transcripts of mRNA may be detected after capping and/or tailing of the synthesized mRNA.

In some embodiments, the full-length mRNA molecule is at least 100 bases, 200 bases, 300 bases, 400 bases, 500 bases, 600 bases, 700 bases, 800 bases, 900 bases, 1 kb, 1.5 kb, 2 kb, 2.5 kb, 3 kb, 3.5 kb, 4 kb, 4.5 kb, 5 kb, 6 kb, 8 kb, 10 kb, 12 kb, 14 kb, 15 kb, 18 kb, or 20 kb in length.

In some embodiments, at least 200 mg, 300 mg, 400 mg, 500 mg, 600 mg, 700 mg, 800 mg, 900 mg, 1 g, 5 g, 10 g, 25 g, 50 g, 75 g, 100 g, 150 g, 200 g, 250 g, 500 g, 750 g, 1 kg, 5 kg, 10 kg, 50 kg, 100 kg, 1000 kg, or more of mRNA is synthesized and purified in a single batch.

In some embodiments, the purified mRNA is assessed for one or more of the following characteristics: appearance, identity, quantity, concentration, presence of impurities, microbiological assessment, pH level and activity. In some embodiments, acceptable appearance includes a clear, colorless solution, essentially free of visible particulates. In some embodiments, the identity of the mRNA is assessed by sequencing methods. In some embodiments, the concentration is assessed by a suitable method, such as UV spectrophotometry. In some embodiments, a suitable concentration is between about 90% and 110% nominal (0.9-1.1 mg/mL).

In some embodiments, assessing the purity of the mRNA includes assessment of mRNA integrity, assessment of residual plasmid DNA, and assessment of residual solvent. In some embodiments, acceptable levels of mRNA integrity are assessed by agarose gel electrophoresis. The gels are analyzed to determine whether the banding pattern and apparent nucleotide length is consistent with an analytical reference standard. Additional methods to assess RNA integrity include, for example, assessment of the purified mRNA using capillary gel electrophoresis (CGE). In some embodiments, acceptable purity of the purified mRNA as determined by CGE is that the purified mRNA composition has no greater than about 70% long abortive/degraded species. In some embodiments, residual plasmid DNA is assessed by methods in the art, for example by the use of qPCR. In some embodiments, less than 10 pg/mg (e.g., less than 10 pg/mg, less than 9 pg/mg, less than 8 pg/mg, less than 7 pg/mg, less than 6 pg/mg, less than 5 pg/mg, less than 4 pg/mg, less than 3 pg/mg, less than 2 pg/mg, or less than 1 pg/mg) is an acceptable level of residual plasmid DNA. In some embodiments, acceptable residual solvent levels are not more than 10,000 ppm, 9,000 ppm, 8,000 ppm, 7,000 ppm, 6,000 ppm, 5,000 ppm, 4,000 ppm, 3,000 ppm, 2,000 ppm, 1,000 ppm. Accordingly, in some embodiments, acceptable residual solvent levels are not more than 10,000 ppm. In some embodiments, acceptable residual solvent levels are not more than 9,000 ppm. In some embodiments, acceptable residual solvent levels are not more than 8,000 ppm. In some embodiments, acceptable residual solvent levels are not more than 7,000 ppm. In some embodiments, acceptable residual solvent levels are not more than 6,000 ppm. In some embodiments, acceptable residual solvent levels are not more than 5,000 ppm. In some embodiments, acceptable residual solvent levels are not more than 4,000 ppm. In some embodiments, acceptable residual solvent levels are not more than 3,000 ppm. In some embodiments, acceptable residual solvent levels are not more than 2,000 ppm. In some embodiments, acceptable residual solvent levels are not more than 1,000 ppm.

In some embodiments, microbiological tests are performed on the purified mRNA, which include, for example, assessment of bacterial endotoxins. In some embodiments, bacterial endotoxins are <0.5 EU/mL, <0.4 EU/mL, <0.3 EU/mL, <0.2 EU/mL or <0.1 EU/mL. Accordingly, in some embodiments, bacterial endotoxins in the purified mRNA are <0.5 EU/mL. In some embodiments, bacterial endotoxins in the purified mRNA are <0.4 EU/mL. In some embodiments, bacterial endotoxins in the purified mRNA are <0.3 EU/mL. In some embodiments, bacterial endotoxins in the purified mRNA are <0.2 EU/mL. In some embodiments, bacterial endotoxins in the purified mRNA are <0.2 EU/mL. In some embodiments, bacterial endotoxins in the purified mRNA are <0.1 EU/mL. In some embodiments, the purified mRNA has not more than 1 CFU/10 mL, 1 CFU/25 mL, 1 CFU/50 mL, 1 CFU/75 mL, or not more than 1 CFU/100 mL. Accordingly, in some embodiments, the purified mRNA has not more than 1 CFU/10 mL. In some embodiments, the purified mRNA has not more than 1 CFU/25 mL. In some embodiments, the purified mRNA has not more than 1 CFU/50 mL. In some embodiments, the purified mRNA has not more than 1 CFR/75 mL. In some embodiments, the purified mRNA has 1 CFU/100 mL.

In some embodiments, the pH of the purified mRNA is assessed. In some embodiments, acceptable pH of the purified mRNA is between 5 and 8. Accordingly, in some embodiments, the purified mRNA has a pH of about 5. In some embodiments, the purified mRNA has a pH of about 6. In some embodiments, the purified mRNA has a pH of about 7. In some embodiments, the purified mRNA has a pH of about 7. In some embodiments, the purified mRNA has a pH of about 8.

In some embodiments, the translational fidelity of the purified mRNA is assessed. The translational fidelity can be assessed by various methods and include, for example, transfection and Western blot analysis. Acceptable characteristics of the purified mRNA includes banding pattern on a Western blot that migrates at a similar molecular weight as a reference standard.

In some embodiments, the purified mRNA is assessed for conductance. In some embodiments, acceptable characteristics of the purified mRNA include a conductance of between about 50% and 150% of a reference standard.

The purified mRNA is also assessed for Cap percentage and for PolyA tail length. In some embodiments, an acceptable Cap percentage includes Cap1, % Area: NLT90. In some embodiments, an acceptable PolyA tail length is about 100-1500 nucleotides (e.g., 100, 150, 200, 250, 300, 350, 400, 450, 500, 550, 600, 650, 700, 750, 800, 850, 900, 950, and 1000, 1100, 1200, 1300, 1400, or 1500 nucleotides).

In some embodiments, the purified mRNA is also assessed for any residual PEG. In some embodiments, the purified mRNA has less than between 10 ng PEG/mg of purified mRNA and 1000 ng PEG/mg of mRNA. Accordingly, in some embodiments, the purified mRNA has less than about 10 ng PEG/mg of purified mRNA. In some embodiments, the purified mRNA has less than about 100 ng PEG/mg of purified mRNA. In some embodiments, the purified mRNA has less than about 250 ng PEG/mg of purified mRNA. In some embodiments, the purified mRNA has less than about 500 ng PEG/mg of purified mRNA. In some embodiments, the purified mRNA has less than about 750 ng PEG/mg of purified mRNA. In some embodiments, the purified mRNA has less than about 1000 ng PEG/mg of purified mRNA.

Various methods of detecting and quantifying mRNA purity are known in the art. For example, such methods include, blotting, capillary electrophoresis, chromatography, fluorescence, gel electrophoresis, HPLC, silver stain, spectroscopy, ultraviolet (UV), or UPLC, or a combination thereof. In some embodiments, mRNA is first denatured by a Glyoxal dye before gel electrophoresis (“Glyoxal gel electrophoresis”). In some embodiments, synthesized mRNA is characterized before capping or tailing. In some embodiments, synthesized mRNA is characterized after capping and tailing.

Capping and Tailing Efficiencies

The purified mRNA is also assessed for Cap percentage and for Poly-A tail length. In some embodiments, an acceptable Cap percentage includes Cap1, % Area: NLT90. Various methods known in the art can be used to assess capping and tailing efficiency and tail length. In some embodiments, capping efficiency is assessed by UPLC-MS Cap assay. In some embodiments, tailing efficiency is assessed by capillary electrophoresis (CE) shift. In some embodiments, RNA tail length is assessed by CE shirt. In some embodiments, RNA tail length is assessed by agarose gel electrophoresis.

In some embodiments, an acceptable Poly-A tail length is about 100-1500 nucleotides (e.g., 100, 150, 200, 250, 300, 350, 400, 450, 500, 550, 600, 650, 700, 750, 800, 850, 900, 950, and 1000, 1100, 1200, 1300, 1400, or 1500 nucleotides). Accordingly, in some embodiments an acceptable Poly-A tail length is about 100 nucleotides. In some embodiments, an acceptable Poly-A tail length is about 200 nucleotides. In some embodiments, an acceptable Poly-A tail length is about 250 nucleotides. In some embodiments, an acceptable Poly-A tail length is about 300 nucleotides. In some embodiments, an acceptable Poly-A tail length is about 350 nucleotides. In some embodiments, an acceptable Poly-A tail length is about 400 nucleotides. In some embodiments, an acceptable Poly-A tail length is about 450 nucleotides. In some embodiments, an acceptable Poly-A tail length is about 500 nucleotides. In some embodiments, an acceptable Poly-A tail length is about 550 nucleotides. In some embodiments, an acceptable Poly-A tail length is about 600 nucleotides. In some embodiments, an acceptable Poly-A tail length is about 650 nucleotides. In some embodiments, an acceptable Poly-A tail length is about 700 nucleotides. In some embodiments, an acceptable Poly-A tail length is about 750 nucleotides. In some embodiments, an acceptable Poly-A tail length is about 800 nucleotides. In some embodiments, an acceptable Poly-A tail length is about 850 nucleotides. In some embodiments, an acceptable Poly-A tail length is about 900 nucleotides. In some embodiments, an acceptable Poly-A tail length is about 950 nucleotides. In some embodiments, an acceptable Poly-A tail length is about 1000 nucleotides. In some embodiments, an acceptable Poly-A tail length is about 1100 nucleotides. In some embodiments, an acceptable Poly-A tail length is about 1200 nucleotides. In some embodiments, an acceptable Poly-A tail length is about 1300 nucleotides. In some embodiments, an acceptable Poly-A tail length is about 1400 nucleotides. In some embodiments, an acceptable Poly-A tail length is about 1500 nucleotides.

Scale

A particular advantage provided by the present invention is the ability to prepare mRNA, in particular, mRNA synthesized in vitro, at a large or commercial scale. For example, in some embodiments in vitro synthesized mRNA is prepared at a scale of or greater than about 100 milligram, 1 gram, 10 gram, 50 gram, 150 gram, 100 gram, 150 gram, 200 gram, 250 gram, 300 gram, 350 gram, 400 gram, 450 gram, 500 gram, 550 gram, 600 gram, 650 gram, 700 gram, 750 gram, 800 gram, 850 gram, 900 gram, 1 kg, 5 kg, 10 kg, 50 kg, 100 kg, one metric ton, ten metric ton or more per batch. In embodiments, in vitro synthesized mRNA is prepared at a scale of or greater than about 1 kg.

In one particular embodiment, in vitro synthesized mRNA is prepared at a scale of 10 gram per batch. In one particular embodiment, in vitro synthesized mRNA is prepared at a scale of 20 gram per batch. In one particular embodiment, in vitro synthesized mRNA is prepared at a scale of 25 gram per batch. In one particular embodiment, in vitro synthesized mRNA is prepared at a scale of 50 gram per batch. In another particular embodiment, in vitro synthesized mRNA is prepared at a scale of 100 gram per batch. In another particular embodiment, in vitro synthesized mRNA is prepared at a scale of 250 gram per batch. In yet another particular embodiment, in vitro synthesized mRNA is prepared at a scale of 1 kg per batch. In yet another particular embodiment, in vitro synthesized mRNA is prepared at a scale of 10 kg per batch. In yet another particular embodiment, in vitro synthesized mRNA is prepared at a scale of 100 kg per batch. In yet another particular embodiment, in vitro synthesized mRNA is prepared at a scale of 1,000 kg per batch. In yet another particular embodiment, in vitro synthesized mRNA is prepared at a scale of 10,000 kg per batch.

In some embodiments, the mRNA is prepared at a scale of or greater than 1 gram, 5 gram, 10 gram, 15 gram, 20 gram, 25 gram, 30 gram, 35 gram, 40 gram, 45 gram, 50 gram, 75 gram, 100 gram, 150 gram, 200 gram, 250 gram, 300 gram, 350 gram, 400 gram, 450 gram, 500 gram, 550 gram, 600 gram, 650 gram, 700 gram, 750 gram, 800 gram, 850 gram, 900 gram, 950 gram, 1 kg, 2.5 kg, 5 kg, 7.5 kg, 10 kg, 25 kg, 50 kg, 75 kg, 100 kg or more per batch.

In some embodiments, the solution comprising mRNA includes at least one gram, ten grams, one-hundred grams, one kilogram, ten kilograms, one-hundred kilograms, one metric ton, ten metric tons, or more mRNA, or any amount there between. In some embodiments, a method described herein is used to prepare an amount of mRNA that is at least about 250 mg mRNA. In one embodiment, a method described herein is used to prepare an amount of mRNA that is at least about 250 mg mRNA, about 500 mg mRNA, about 750 mg mRNA, about 1000 mg mRNA, about 1500 mg mRNA, about 2000 mg mRNA, or about 2500 mg mRNA. In embodiments, a method described herein is used to prepare an amount of mRNA that is at least about 250 mg mRNA to about 500 g mRNA. In embodiments, a method described herein is used to prepare an amount of mRNA that is at least about 500 mg mRNA to about 250 g mRNA, about 500 mg mRNA to about 100 g mRNA, about 500 mg mRNA to about 50 g mRNA, about 500 mg mRNA to about 25 g mRNA, about 500 mg mRNA to about 10 g mRNA, or about 500 mg mRNA to about 5 g mRNA. In embodiments, a method described herein is used to prepare an amount of mRNA that is at least about 100 mg mRNA to about 10 g mRNA, about 100 mg mRNA to about 5 g mRNA, or about 100 mg mRNA to about 1 g mRNA.

Yield

In some embodiments, a method described herein provides a recovered amount of purified mRNA (or “yield”) that is at least about 40%, 45%, 50%, about 55%, about 60%, about 65%, about 70%, about 75%, about 80%, about 85%, about 90%, about 95% about 97%, about 98%, about 99%, or about 100%. Accordingly, in some embodiments, the recovered amount of purified mRNA is about 40%. In some embodiments, the recovered amount of purified mRNA is about 45%. In some embodiments, the recovered amount of purified mRNA is about 50%. In some embodiments, the recovered amount of purified mRNA is about 55%. In some embodiments, the recovered amount of purified mRNA is about 60%. In some embodiments, the recovered amount of purified mRNA is about 65%. In some embodiments, the recovered amount of purified mRNA is about 70%. In some embodiments, the recovered amount of purified mRNA is about 75%. In some embodiments, the recovered amount of purified mRNA is about 75%. In some embodiments, the recovered amount of purified mRNA is about 80%. In some embodiments, the recovered amount of purified mRNA is about 85%. In some embodiments, the recovered amount of purified mRNA is about 90%. In some embodiments, the recovered amount of purified mRNA is about 91%. In some embodiments, the recovered amount of purified mRNA is about 92%. In some embodiments, the recovered amount of purified mRNA is about 93%. In some embodiments, the recovered amount of purified mRNA is about 94%. In some embodiments, the recovered amount of purified mRNA is about 95%. In some embodiments, the recovered amount of purified mRNA is about 96%. In some embodiments, the recovered amount of purified mRNA is about 97%. In some embodiments, the recovered amount of purified mRNA is about 98%. In some embodiments, the recovered amount of purified mRNA is about 99%. In some embodiments, the recovered amount of purified mRNA is about 100%.

Purity

The mRNA composition described herein is substantially free of contaminants comprising short abortive RNA species, long abortive RNA species, double-stranded RNA (dsRNA), residual plasmid DNA, residual in vitro transcription enzymes, residual solvent and/or residual salt.

The mRNA composition described herein has a purity of about between 60% and about 100%. Accordingly, in some embodiments, the purified mRNA has a purity of about 60%. In some embodiments, the purified mRNA has a purity of about 65%. In some embodiments, the purified mRNA has a purity of about 70%. In some embodiments, the purified mRNA has a purity of about 75%. In some embodiments, the purified mRNA has a purity of about 80%. In some embodiments, the purified mRNA has a purity of about 85%. In some embodiments, the purified mRNA has a purity of about 90%. In some embodiments, the purified mRNA has a purity of about 91%. In some embodiments, the purified mRNA has a purity of about 92%. In some embodiments, the purified mRNA has a purity of about 93%. In some embodiments, the purified mRNA has a purity of about 94%. In some embodiments, the purified mRNA has a purity of about 95%. In some embodiments, the purified mRNA has a purity of about 96%. In some embodiments, the purified mRNA has a purity of about 97%. In some embodiments, the purified mRNA has a purity of about 98%. In some embodiments, the purified mRNA has a purity of about 99%. In some embodiments, the purified mRNA has a purity of about 100%.

In some embodiments, the mRNA composition described herein has less than 10%, less than 9%, less than 8%, less than 7%, less than 6%, less than 5%, less than 4%, less than 3%, less than 2%, less than 1%, less than 0.5%, and/or less than 0.1% impurities other than full-length mRNA. The impurities include IVT contaminants, e.g., proteins, enzymes, DNA templates, free nucleotides, residual solvent, residual salt, double-stranded RNA (dsRNA), prematurely aborted RNA sequences (“shortmers” or “short abortive RNA species”), and/or long abortive RNA species. In some embodiments, the purified mRNA is substantially free of process enzymes.

In some embodiments, the residual plasmid DNA in the purified mRNA of the present invention is less than about 1 pg/mg, less than about 2 pg/mg, less than about 3 pg/mg, less than about 4 pg/mg, less than about 5 pg/mg, less than about 6 pg/mg, less than about 7 pg/mg, less than about 8 pg/mg, less than about 9 pg/mg, less than about 10 pg/mg, less than about 11 pg/mg, or less than about 12 pg/mg. Accordingly, the residual plasmid DNA in the purified mRNA is less than about 1 pg/mg. In some embodiments, the residual plasmid DNA in the purified mRNA is less than about 2 pg/mg. In some embodiments, the residual plasmid DNA in the purified mRNA is less than about 3 pg/mg. In some embodiments, the residual plasmid DNA in the purified mRNA is less than about 4 pg/mg. In some embodiments, the residual plasmid DNA in the purified mRNA is less than about 5 pg/mg. In some embodiments, the residual plasmid DNA in the purified mRNA is less than about 6 pg/mg. In some embodiments, the residual plasmid DNA in the purified mRNA is less than about 7 pg/mg. In some embodiments, the residual plasmid DNA in the purified mRNA is less than about 8 pg/mg. In some embodiments, the residual plasmid DNA in the purified mRNA is less than about 9 pg/mg. In some embodiments, the residual plasmid DNA in the purified mRNA is less than about 10 pg/mg. In some embodiments, the residual plasmid DNA in the purified mRNA is less than about 11 pg/mg. In some embodiments, the residual plasmid DNA in the purified mRNA is less than about 12 pg/mg.

In some embodiments, a method according to the invention removes more than about 90%, 95%, 96%, 97%, 98%, 99% or substantially all prematurely aborted RNA sequences (also known as “shortmers”). In some embodiments, mRNA composition is substantially free of prematurely aborted RNA sequences. In some embodiments, mRNA composition contains less than about 5% (e.g., less than about 4%, 3%, 2%, or 1%) of prematurely aborted RNA sequences. In some embodiments, mRNA composition contains less than about 1% (e.g., less than about 0.9%, 0.8%, 0.7%, 0.6%, 0.5%, 0.4%, 0.3%, 0.2%, or 0.1%) of prematurely aborted RNA sequences. In some embodiments, mRNA composition undetectable prematurely aborted RNA sequences as determined by, e.g., high-performance liquid chromatography (HPLC) (e.g., shoulders or separate peaks), ethidium bromide, Coomassie staining, capillary electrophoresis or Glyoxal gel electrophoresis (e.g., presence of separate lower band). As used herein, the term “shortmers”, “short abortive RNA species”, “prematurely aborted RNA sequences” or “long abortive RNA species” refers to any transcripts that are less than full-length. In some embodiments, “shortmers”, “short abortive RNA species”, or “prematurely aborted RNA sequences” are less than 100 nucleotides in length, less than 90, less than 80, less than 70, less than 60, less than 50, less than 40, less than 30, less than 20, or less than 10 nucleotides in length. In some embodiments, shortmers are detected or quantified after adding a 5′-cap, and/or a 3′-poly A tail. In some embodiments, prematurely aborted RNA transcripts comprise less than 15 bases (e.g., less than 14, 13, 12, 11, 10, 9, 8, 7, 6, 5, 4, or 3 bases). In some embodiments, the prematurely aborted RNA transcripts contain about 8-15, 8-14, 8-13, 8-12, 8-11, or 8-10 bases.

In some embodiments, a purified mRNA of the present invention is substantially free of enzyme reagents used in in vitro synthesis including, but not limited to, T7 RNA polymerase, DNAse I, pyrophosphatase, and/or RNAse inhibitor. In some embodiments, a purified mRNA according to the present invention contains less than about 5% (e.g., less than about 4%, 3%, 2%, or 1%) of enzyme reagents used in in vitro synthesis including. In some embodiments, a purified mRNA contains less than about 1% (e.g., less than about 0.9%, 0.8%, 0.7%, 0.6%, 0.5%, 0.4%, 0.3%, 0.2%, or 0.1%) of enzyme reagents used in in vitro synthesis including. In some embodiments, a purified mRNA contains undetectable enzyme reagents used in in vitro synthesis including as determined by, e.g., silver stain, gel electrophoresis, high-performance liquid chromatography (HPLC), ultra-performance liquid chromatography (UPLC), and/or capillary electrophoresis, ethidium bromide and/or Coomassie staining.

### Therapeutic Use of Compositions

The mRNAs prepared according to methods of the present invention can be used as a drug product for therapeutic use. Particularly, the mRNAs prepared according to methods of the present invention can be delivered to subjects in need of for in vivo protein production. To facilitate expression of mRNA in vivo, delivery vehicles such as liposomes can be formulated in combination with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition.

In some embodiments, a composition comprises mRNA encapsulated or complexed with a delivery vehicle. In some embodiments, the delivery vehicle is selected from the group consisting of liposomes, lipid nanoparticles, solid-lipid nanoparticles, polymers, viruses, sol-gels, and nanogels.

In some embodiments, a suitable delivery vehicle is a liposomal delivery vehicle, e.g., a lipid nanoparticle. As used herein, liposomal delivery vehicles, e.g., lipid nanoparticles, are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). In the context of the present invention, a liposomal delivery vehicle typically serves to transport a desired nucleic acid (e.g., mRNA or MCNA) to a target cell or tissue.

In some embodiments, a nanoparticle delivery vehicle is a liposome. In some embodiments, a liposome comprises one or more cationic lipids, one or more non-cationic lipids, one or more cholesterol-based lipids, or one or more PEG-modified lipids. A typical liposome for use with the invention is composed of four lipid components: a cationic lipid, a non-cationic lipid (e.g., DOPE or DEPE), a cholesterol-based lipid (e.g., cholesterol) and a PEG-modified lipid (e.g., DMG-PEG2K). In some embodiments, a liposome comprises no more than three distinct lipid components. In some embodiments, one distinct lipid component is a sterol-based cationic lipid. An exemplary liposome is composed of three lipid components: a sterol-based cationic lipid, a non-cationic lipid (e.g., DOPE or DEPE) and a PEG-modified lipid (e.g., DMG-PEG2K).

Various methods for encapsulating mRNA are described in published U.S. Application No. US 2011/0244026, published U.S. Application No. US 2016/0038432, published U.S. Application No. US 2018/0153822, published U.S. Application No. US 2018/0125989 and U.S. Provisional Application No. 62/877,597, filed Jul. 23, 2019 and can be used to practice the present invention, all of which are incorporated herein by reference.

### EXAMPLES

### Example 1. Synthesis and Analysis of Capped and Tailed mRNA

In Vitro Transcription mRNA Synthesis

In the following examples, unless otherwise described, mRNA was synthesized via in vitro transcription (IVT) using either T7 polymerase of SP6 polymerase. Any method of IVT synthesis known in the art can be used to practice the invention. The in vitro transcribed mRNA was purified, concentrated via ultrafiltration/diafiltration (UFDF) prior to cap/tail reaction.

The purified mRNA product from the aforementioned in vitro transcription step was capped with Cap1 and tailed. The reaction mixture was treated with portions of GTP (1.0 mM), S-adenosyl methionine, RNAse inhibitor, 2′O-Methyltransferase and guanylyl transferase are mixed together with reaction buffer (10×, 500 mM Tris-HCl (pH 8.0 or pH 7.5), 60 mM KCl, MgCl₂ at 12.5 or 10.0 mM). The combined solution was incubated for a range of time at 37° C. for 30 to 90 minutes. Upon completion, aliquots of ATP (2.0 mM), PolyA Polymerase and tailing reaction buffer were added and the total reaction mixture was further incubated at 37° C. for a range of time from 20 to 45 minutes. Upon completion, the final reaction mixture was quenched and purified accordingly.

RNA Integrity Analysis (Fragment Analyzer—Capillary Electrophoresis)

RNA integrity and tail length were assessed using a capillary electrophoresis (CE) fragment analyzer and the commercially available RNA detection kit. Analysis of peak profiles for integrity and size shift for tail length were performed on raw data as well as normalized data sets.

mRNA Cap Species Analysis (HPLC/MS)

Cap species present in the final purified mRNA product were quantified using the chromatographic method described in U.S. Pat. No. 9,970,047. This method is capable of accurately quantifying uncapped mRNA as a percent of total mRNA. This method also can quantify amounts of particular cap structures, such as CapG, Cap0 and Cap 1 amounts, which can be reported as a percentage of total mRNA.

### Example 2. Optimized Cap and Tail Reaction Condition Increases CFTR mRNA Integrity

This example illustrates that cap and tail reaction condition of the present invention provides an increased mRNA integrity suitable for therapeutic use. The increased mRNA integrity was independent of mRNA construct size or nucleotide composition.

CFTR mRNA (˜4,600 nt) and DNAH5 mRNA (˜14,000 nt) were synthesized via IVT synthesis and purified as described in Example 1. Prior to the cap and tail reaction, the purified mRNAs were analyzed using CE. 5 mg batches of the purified and concentrated IVT mRNAs were then capped and tailed via an enzymatic step in two different conditions as shown in Table 1. Other than the concentration of MgCl₂ and pH, the rest of the reaction condition variables remained the same. The integrity and poly A tail length of purified capped and tailed mRNAs was assessed by CE as described in Example 1.

TABLE 1
Cap and tail reaction conditions

| Sample | mRNA | MgCl₂ (mM) | Buffer | Scale |
|---|---|---|---|---|
| A | CFTR | 1.25 | 50 mM Tris pH 8.0 | 5 mg |
| B | CFTR | 1.0 | 50 mM Tris pH 7.5 | 5 mg |
| C | DNAH5 | 1.25 | 50 mM Tris pH 8.0 | 5 mg |
| D | DNAH5 | 1.0 | 50 mM Tris pH 7.5 | 5 mg |

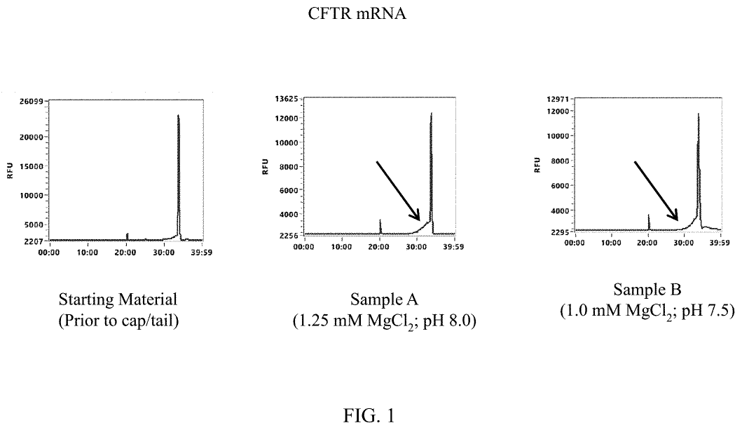

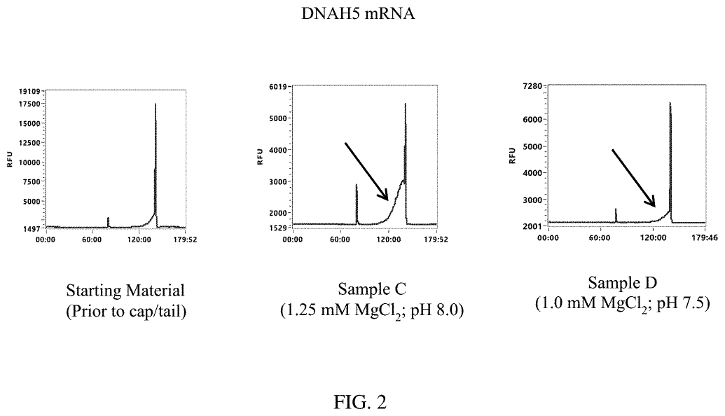

For both CFTR and DNAH5 mRNAs, optimized cap and tail reaction conditions resulted in an increase in mRNA integrity as compared to control. As shown in FIG. 1, the final product of capped/tailed CFTR mRNA in optimized condition (Sample B) has a well-defined peak with the tail length within the target range. Sample B, which was capped and tailed in a reaction condition comprising 1.0 mM MgCl₂ and 50 mM Tris at pH 7.5, was substantially free of the “shoulder” (indicated by arrows in FIG. 1). Similarly, FIG. 2 shows that the final product of sample D has well-defined peak with the tail length within the target range. Notably, DNAH5 mRNA capped and tailed in a reaction condition comprising 1.0 mM MgCl₂ and 50 mM Tris at pH 7.5 showed a more intense and sharper peak corresponding to the full-length product and was substantially free of the “shoulder”. The results demonstrated that the optimized cap and tail reaction conditions of the present invention resulted in an increased RNA integrity regardless of its construct size or nucleotide composition.

### Example 3. Optimized Cap and Tail Reaction at 1-Gram and 15-Gram Scale

This example illustrates that the optimized cap and tail reaction condition of the present invention can be used to cap and tail mRNA at the necessary scale and quality needed for therapeutic use. The mRNA purified at 1- and 15-gram scale according to methods described herein, results in high RNA integrity, capping and tailing efficiency, and desired tail length, demonstrating the scalability of the method.

One batch of CFTR mRNA was synthesized at 1-gram scale, and two batches of CFTR mRNA were synthesized at 15-gram scale via IVT synthesis as described in Example 1. The resulting 1-gram IVT mRNA sample was then capped and tailed via an enzymatic step in reaction condition comprising 50 mM Tris pH 7.5 and 1.0 mM MgCl₂. For 15-gram scale, cap and tail reactions were performed in reaction condition comprising 50 mM Tris pH 8.0 and 1.25 mM MgCl₂ (conventional condition) or 50 mM Tris pH 7.5 and 1.0 mM MgCl₂ (optimized condition). The integrity, tailing efficiency, and poly A tail length of the purified capped and tailed mRNAs was assessed by CE. The capping efficiency was also evaluated by UPLC-MS as described in Example 1.

TABLE 2
Analysis of purified capped and tailed mRNA
at 1-gram scale in optimized condition

| Analytic | Unit | Result |
|---|---|---|
| RNA Integrity(CE Smear) | % Main Peak | 77% |
| Tail Length (CE Shift) | Nucleotides | 487 nt |
| Tailing Efficiency (CE Shift) | % Target Tail Length | 78% |
| Capping Efficiency | % Cap1 | 94% |
| (UPLC-MS Cap Assay) | | |

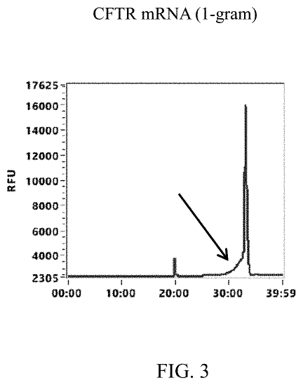

As shown in Table 2, optimized cap and tail reaction condition comprising Tris pH 7.5 and 1.0 mM MgCl₂ resulted in an increase in CFTR mRNA integrity, as well as high capping and tailing efficiency at 1-gram scale. FIG. 3 illustrates that the final product of capped/tailed CFTR mRNA has a well-defined, sharp peak corresponding to the full-length product with the tail length within the target range, and substantially free of the “shoulder.

TABLE 3
Analysis of purified capped and tailed mRNA
at 15-gram scale in optimized condition

| Analytic | Unit | Result |
|---|---|---|
| RNA Integrity(CE Smear) | % Main Peak | 80% |
| RNA Integrity(CGE Smear) | % Main Peak | 71% |
| Tail Length (CE Shift) | Nucleotides | 387 nt |
| Tailing Efficiency (CE Shift) | % Target Tail Length | 77% |
| Capping Efficiency | % Cap1 | 100% |
| (UPLC-MS Cap Assay) | | |

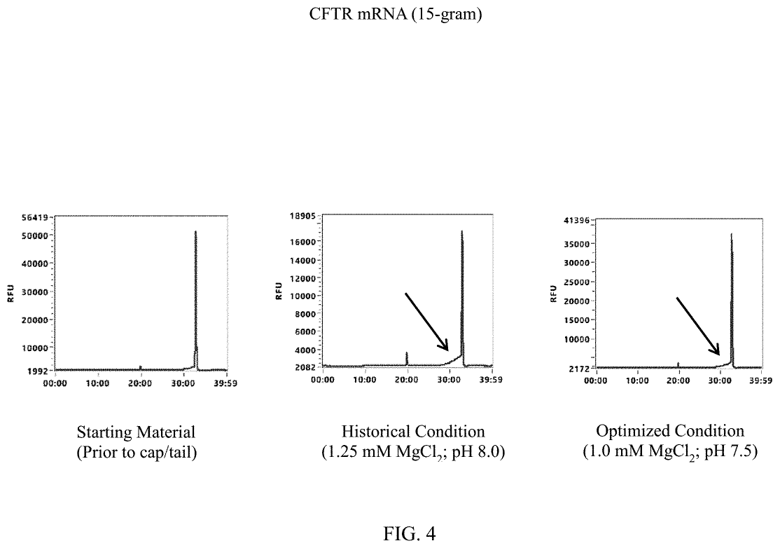

FIG. 4 shows that the final product of CFTR mRNA product has well-defined peak with the tail length within the target range at 15-gram scale. Notably, CFTR mRNA capped and tailed in a reaction condition comprising 1.0 mM MgCl₂ and 50 mM Tris at pH 7.5 (optimized condition) showed a more intense and sharper peak corresponding to the full-length product and was substantially free of the “shoulder”, whereas the shoulder was still visible in CFTR mRNA capped and tailed in historical condition (1.25 mM MgCl₂ at pH 8.0). Analysis also shows that the optimized cap and tail reaction condition comprising Tris pH 7.5 and 1.0 mM MgCl₂ resulted in an increase in CFTR mRNA integrity, as well as high capping and tailing efficiency at 15-gram scale, as shown in Table 3. Notably, RNA integrity was higher than 70% as measured by CE Smear or CGE smear. The poly-A tail length of 387 nt was observed, which was well within the target range of 500 nt. The optimized reaction condition also resulted in tailing efficiency of higher than 75%, and capping efficiency of 100%. Moreover, the capping reaction resulted in 100% Cap1, which was the desired Cap species (Table 4).

TABLE 4
Analysis of Capping Efficiency at 15-
gram scale in optimized condition

| | | % Cap Species | | |
|---|---|---|---|---|
| Sample | Uncapped % | Cap0 | CapG | Cap1 |
| CFTR mRNA | 0 | 0 | 0 | 100 |

Together, the data demonstrate the scalability of the optimized cap and tail reaction condition for mRNA preparation at the necessary scale and quality required for clinical therapeutic use. The capped and tailed mRNAs at 1- and 15-grams scale by methods described herein resulted in a high mRNA integrity while maintaining all other critical quality attributes, demonstrating the method for use in mRNA therapeutics.

### Example 4. Optimized Cap and Tail Reaction at 100-Gram Manufacturing Scale

This example illustrates that the optimized cap and tail reaction condition of the present invention can be used to cap and tail mRNA at manufacturing scale with high RNA integrity. The mRNA purified at 1- and 15-gram scale according to methods described herein, resulted in high integrity, cap and tail efficiency, and desired tail length, demonstrating the scalability of the method.

Two batches of CFTR mRNA was synthesized at 100-gram scale via IVT synthesis as described in Example 1. The resulting 100-gram IVT mRNA samples were then capped and tailed via an enzymatic step in reaction condition comprising 50 mM Tris pH 8.0 and 1.25 mM MgCl₂ or 50 mM Tris pH 7.5 and 1.0 mM MgCl₂. The integrity, tailing efficiency, and poly A tail length of the purified capped and tailed mRNAs was assessed by CE. The capping efficiency was also evaluated by UPLC-MS as described in Example 1.

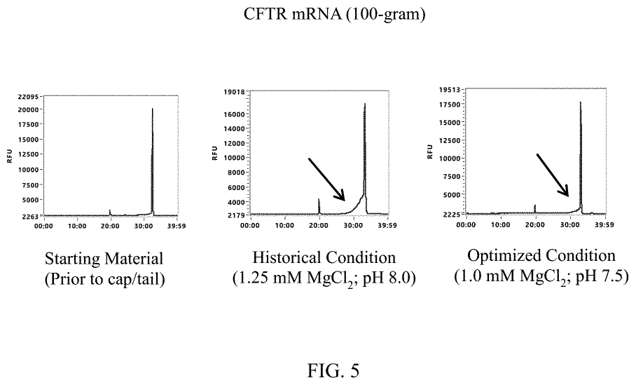

FIG. 5 shows that the final product of CFTR mRNA product has well-defined peak with the tail length within the target range at 100-gram scale. Notably, CFTR mRNA capped and tailed in a reaction condition comprising 1.0 mM MgCl₂ and 50 mM Tris at pH 7.5 (optimized condition) showed a more intense and sharper peak corresponding to the full-length product and was substantially free of the “shoulder”, whereas the shoulder was still visible in CFTR mRNA capped and tailed in historical condition (1.25 mM MgCl₂ at pH 8.0). This demonstrates a significant reduction in degraded RNA species for final mRNA product that was capped and tailed in optimized reaction condition.

Overall, the data demonstrate the scalability of the optimized cap and tail reaction condition for mRNA synthesis at the manufacturing scale and with high quality required for clinical therapeutic use. The capped and tailed mRNAs at 100-gram scale by methods described herein resulted in a high mRNA integrity while maintaining all other critical quality attributes, demonstrating the method for use in mRNA manufacturing and therapeutics.

### Example 5. Optimized Cap and Tail Reaction at 250-Gram Manufacturing Scale

This example illustrates that the optimized cap and tail reaction condition of the present invention can be used to cap and tail mRNA at manufacturing scale with high RNA integrity. The mRNA purified at 1-, 15-gram, 100-gram, and 250-gram scales according to methods described herein, resulted in high integrity, cap and tail efficiency, and desired tail length, demonstrating the scalability of the method.

The OTC mRNA was synthesized at 250-gram scale via IVT synthesis as described in Example 1. The resulting 250-gram IVT mRNA sample was then capped and tailed via an enzymatic step in reaction conditions comprising 50 mM Tris pH 7.5 and 1.0 mM MgCl₂. Another 10-gram IVT mRNA sample was synthesized via IVT synthesis described in Example 1, and capped and tailed via an enzymatic step in reaction conditions comprising 50 mM Tris pH 8.0 and 1.25 mM MgCl₂. The integrity, tailing efficiency, and poly A tail length of the purified capped and tailed mRNAs was assessed by CE. The capping efficiency was also evaluated by UPLC-MS as described in Example 1.

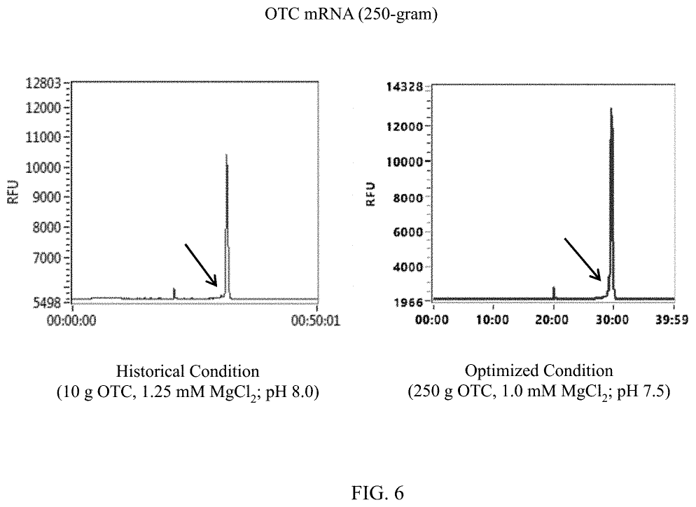

FIG. 6 shows that the final product of OTC mRNA product has a well-defined peak with the tail length within the target range at 250-gram scale. Notably, OTC mRNA capped and tailed in a reaction condition comprising 1.0 mM MgCl₂ and 50 mM Tris at pH 7.5 (optimized condition) showed a more intense and sharper peak corresponding to the full-length product and was substantially free of the “shoulder”, whereas the shoulder was still visible in a 10-gram OTC mRNA sample capped and tailed in historical condition (1.25 mM MgCl₂ at pH 8.0). These results demonstrated that there was a significant reduction in degraded RNA species for final mRNA product that was capped and tailed in optimized reaction conditions.

Overall, the data demonstrated the scalability of the optimized cap and tail reaction condition for mRNA synthesis at the manufacturing scale and with high quality required for clinical therapeutic use. The capped and tailed mRNAs at 250-gram scale by methods described herein resulted in a high mRNA integrity while maintaining all other critical quality attributes, demonstrating the method for use in mRNA manufacturing and therapeutics.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of capping and tailing an in vitro transcribed purified messenger RNA (mRNA) preparation, the method comprising capping and tailing the mRNA in a reaction buffer comprising MgCl₂ and having a pH lower than 8.0.

2. The method of claim 1, wherein the reaction buffer further comprises KCl.

3. (canceled)

4. The method of claim 1, wherein the MgCl₂ in the reaction buffer has a concentration of about 1.0 mM.

5. (canceled)

6. The method of claim 1, wherein the pH of the reaction buffer is between about 7.2 and 7.7.

7. The method of claim 1, wherein the pH of the reaction buffer is about 7.5.

8. The method of claim 1, wherein the mRNA is at a scale of 5 mg, 1 g, 15 g, 100 g, 250 g, 500 g, or 1 kg or above.

9. The method of claim 8, wherein the mRNA is at a scale of 100 g.

10. The method of claim 1, wherein tailing the mRNA comprises addition of a poly-A tail having a length of about between 250 nucleotides and 750 nucleotides.

11. The method of claim 10, wherein tailing the mRNA comprises addition of a poly-A tail having a length of about 500 nucleotides.

12. The method of claim 10, wherein tailing the mRNA has an efficiency of between about 70% and 95%.

13. The method of claim 12, wherein tailing the mRNA has an efficiency of about 80%.

14. The method of claim 1, wherein capping the mRNA has an efficiency of 90% or more.

15. The method of claim 1, where capping the mRNA has an efficiency of about 100%.

16. The method of claim 1, wherein capping and tailing the mRNA in a reaction buffer having a pH lower than 8.0 results in capped and tailed mRNA that has greater integrity in comparison to capped and tailed mRNA using a reaction buffer having a pH of 8.0 or above.

17. The method of claim 1, wherein capping and tailing the mRNA in a reaction buffer having a MgCl₂ concentration of 1.0 mM or less results in a capped and tailed mRNA that has greater integrity in comparison to capped and tailed mRNA using a reaction buffer having a MgCl₂ concentration of greater than 1.0 mM.

18. The method of claim 16, wherein the mRNA integrity is at least 65% or more.

19. The method of claim 18, wherein the mRNA integrity is at least 75% or more.

20. The method of claim 16, wherein the method has an mRNA capping efficiency of 80% or above.

21. The method of claim 20, wherein the mRNA capping efficiency is about 90% or above.

22. A method of capping and tailing an in vitro transcribed purified messenger RNA (mRNA) preparation, the method comprising capping and tailing the mRNA in a reaction buffer comprising a pH of about 7.5, and a MgCl₂ concentration of about 1.0 mM, wherein the capping and tailing of the mRNA has a capping and tailing efficiency of 80% or more, and wherein the capped and tailed mRNA has an integrity of at least 65% or above.
